(19) United States
(12) Patent Application Publication
(10) Pub. No.: US 2021/0100846 A1
(43) Pub. Date: Apr. 8, 2021

(54) USE OF PLATELETS IN TREATING INFECTIONS

(71) Applicant: Cellphire, Inc., Rockville, MD (US)

(72) Inventors: Amber Nicole Lee, Rockville, MD (US); Keith Andrew Moskowitz, Westfield, IN (US)

(21) Appl. No.: 16/931,944

(22) Filed: Jul. 17, 2020

Related U.S. Application Data
(60) Provisional application No. 62/875,055, filed on Jul. 17, 2019.

(51) Int. Cl. A61K 35/19; A61K 9/19; A61K 47/26; A61K 9/00; A61K 47/22; A61K 47/02; A61K 47/42

(52) U.S. Cl. CPC A61K 35/19; A61K 9/19; A61K 47/26; A61K 9/0014; A61K 47/42; A61K 9/007; A61K 47/22; A61K 47/02; A61K 9/0019

(57) ABSTRACT

Provided herein in some embodiments is a method of treating a disease or condition such as an antibiotic resistant bacterial infection, a gram negative bacterial infection, a gram positive bacterial infection, a fungal infection, protozoa, a hemorrhagic virus, such as Ebola or Dengue, or a non-hemorrhagic virus, such as a coronavirus, in a subject, comprising administering to the subject in need thereof an effective amount of a composition comprising platelets or platelet derivatives, such as freeze-dried platelets; and an incubating agent comprising one or more salts, a buffer, optionally a cryoprotectant, and optionally an organic solvent.

### CROSS-REFERENCE TO RELATED APPLICATIONS

This application claims priority to U.S. Provisional Application Ser. No. 62/875,055 filed on Jul. 17, 2019.

### TECHNICAL FIELD

The present disclosure in some embodiments relates to the use of platelets, platelet derivatives, or thrombosomes to bind foreign bodies in a subject, such as pathogens, such as bacteria in the bloodstream of the subject, such as Staphylococcus aureus. In some embodiments, the foreign bodies bound to the platelets, platelet derivatives, or thrombosomes may be removed from circulation upon natural clearance of the platelets, platelet derivatives, or thrombosomes or may be acted on by an immune response.

The present disclosure relates to the field of blood and blood products. More specifically, it relates to platelets, cryopreserved platelets, and/or lyopreserved platelet compositions, including those containing stabilized platelets or compositions derived from platelets. The platelets can be stored under typical ambient conditions, refrigerated, cryopreserved, for example with dimethyl sulfoxide (DMSO), and/or lyophilized after stabilization (e.g., thrombosomes),

### DESCRIPTION OF RELATED ART

Blood is a complex mixture of numerous components. In general, blood can be described as comprising four main parts: red blood cells, white blood cells, platelets, and plasma. The first three are cellular or cell-like components, whereas the fourth (plasma) is a liquid component comprising a wide and variable mixture of salts, proteins, and other factors necessary for numerous bodily functions. The components of blood can be separated from each other by various methods. In general, differential centrifugation is most commonly used currently to separate the different components of blood based on size and, in some applications, density.

Unactivated platelets, which are also commonly referred to as thrombocytes, are small, often irregularly-shaped (e.g., discoidal or ovoidal) megakaryocyte-derived components of blood that are involved in the clotting process. They aid in protecting the body from excessive blood loss due not only to trauma or injury, but to normal physiological activity as well. Platelets are considered crucial in normal hemostasis, providing the first line of defense against blood escaping from injured blood vessels. Platelets generally function by adhering to the lining of broken blood vessels, in the process becoming activated, changing to an amorphous shape, and interacting with components of the clotting system that are present in plasma or are released by the platelets themselves or other components of the blood. Purified platelets have found use in treating subjects with low platelet count (thrombocytopenia) and abnormal platelet function (thrombasthenia). Concentrated platelets are often used to control bleeding after injury or during acquired platelet function defects or deficiencies, for example those occurring during surgery and those due to the presence of platelet inhibitors.

Current treatments for sepsis include: antibiotics and steroids to fight infection; supportive care to increase blood pressure and prevent dehydration. In cases of kidney failure, patients may need dialysis. There is no widely practiced clinical method for physically removing infectious particles from the circulation.

### SUMMARY OF THE INVENTION

Provided herein in some embodiments is a method of treating a disease or condition selected from an antibiotic resistant bacterial infection, a gram negative bacterial infection, a gram positive bacterial infection, a fungal infection, protozoan infection, a hemorrhagic virus, such as Ebola or Dengue, or a non-hemorrhagic virus, in a subject, comprising administering to the subject in need thereof an effective amount of a composition comprising platelets or platelet derivatives, such as freeze-dried platelets; and an incubating agent comprising one or more salts, a buffer, optionally a cryoprotectant (also called a lyophilizing agent), and optionally an organic solvent.

In some embodiments provided herein is a method of treating an antibiotic resistant bacterial infection in a subject, the method comprising administering to the subject in need thereof an effective amount of a composition comprising platelets or platelet derivatives, such as freeze-dried platelets; and an incubating agent comprising one or more salts, a buffer, optionally a cryoprotectant, and optionally an organic solvent.

In some embodiments provided herein is a method of treating a gram negative bacterial infection in a subject, the method comprising administering to the subject in need thereof an effective amount of a composition comprising platelets or platelet derivatives, such as freeze-dried platelets; and an incubating agent comprising one or more salts, a buffer, optionally a cryoprotectant, and optionally an organic solvent.

In some embodiments provided herein is a method of treating a gram positive bacterial infection in a subject, the method comprising administering to the subject in need thereof an effective amount of a composition comprising platelets or platelet derivatives, such as freeze-dried platelets; and an incubating agent comprising one or more salts, a buffer, optionally a cryoprotectant, and optionally an organic solvent.

In some embodiments provided herein is a method of treating a fungal infection in a subject, the method comprising administering to the subject in need thereof an effective amount of a composition comprising platelets or platelet derivatives, such as freeze-dried platelets and an incubating agent comprising one or more salts, a buffer, optionally a cryoprotectant, and optionally an organic solvent.

In some embodiments provided herein is a method of treating protozoa in a subject, the method comprising administering to the subject in need thereof an effective amount of a composition comprising platelets or platelet derivatives, such as freeze-dried platelets; and an incubating agent comprising one or more salts, a buffer, optionally a cryoprotectant, and optionally an organic solvent.

In some embodiments provided herein is a method of treating a hemorrhagic virus, such as Ebola or Dengue, in a subject, the method comprising administering to the subject in need thereof an effective amount of a composition comprising platelets or platelet derivatives, such as freeze-dried platelets; and an incubating agent comprising one or more salts, a buffer, optionally a cryoprotectant, and optionally an organic solvent.

In some embodiments provided herein is a method of treating a non-hemorrhagic virus in a subject, the method comprising administering to the subject in need thereof an effective amount of a composition comprising platelets or platelet derivatives, such as freeze-dried platelets; and an incubating agent comprising one or more salts, a buffer, optionally a cryoprotectant, and optionally an organic solvent.

In some embodiments provided herein is a method of binding a foreign body in a subject, such as pathogens, such as bacteria in the bloodstream of the subject, such as Staphylococcus aureus, the method comprising administering to the subject in need thereof an effective amount of a composition comprising platelets or platelet derivatives, such as freeze-dried platelets; and an incubating agent comprising one or more salts, a buffer, optionally a cryoprotectant, and optionally an organic solvent, wherein the foreign body binds to the platelets or platelet derivatives, such as freeze-dried platelets.

In some embodiments provided herein is a method of binding a foreign body in the bloodstream of a subject, such as pathogens, such as bacteria in the bloodstream of the subject, such as Staphylococcus aureus, the method comprising administering to the subject in need thereof an effective amount of a composition comprising platelets or platelet derivatives, such as freeze-dried platelets; and an incubating agent comprising one or more salts, a buffer, optionally a cryoprotectant, and optionally an organic solvent, wherein the amount of the foreign body in the bloodstream of the subject decreases by at least 5%.

In some embodiments provided herein is an in vitro method of detecting an interaction between platelets or platelet derivatives and an incubating agent comprising one or more salts, a buffer, optionally a cryoprotectant and a foreign body, the method comprising combining a composition comprising platelets or platelet derivatives and an incubating agent comprising one or more salts, a buffer, optionally a cryoprotectant, and optionally an organic solvent, a foreign body, and an aqueous medium, and detecting an interaction between the composition and the foreign body.

### DESCRIPTION OF DRAWINGS

FIG. 1 is a graph plotting light transmission aggregometry results as for fresh platelets as described in Example 1.

FIG. 2 is a graph plotting light transmission aggregometry results for stored platelets as described in Example 1.

FIG. 3 is a graph plotting light transmission aggregometry results for thrombosomes as described in Example 1.

FIG. 4 is a bar chart plotting maximum aggregation (by percent) for Example 1.

FIG. 5 is a bar chart plotting the raw decrease in optical density for Example 1.

FIG. 6A is an exemplary flow cytometry plot of BODIPY-labeled PANSORBIN® in supplemented HTMA.

FIG. 6B is an exemplary flow cytometry plot of Streptavidin-Dylight633-labeled thrombosomes in supplemented HTMA.

FIG. 6C is an exemplary flow cytometry plot of a mixture of BODIPY-labeled PANSORBIN® and Streptavidin-Dylight633-labeled thrombosomes in supplemented HTMA.

FIG. 7A is an exemplary flow cytometry plot of BODIPY-labeled PANSORBIN® in non-supplemented HTMA

FIG. 7B is an exemplary flow cytometry plot of Streptavidin-Dylight633-labeled thrombosomes in non-supplemented HTMA.

FIG. 7C is an exemplary flow cytometry plot of a mixture of BODIPY-labeled PANSORBIN® and Streptavidin-Dylight633-labeled thrombosomes in non-supplemented HTMA.

FIG. 8A is an exemplary fluorescence microscopy image of a mixture of BODIPY-labeled PANSORBIN® and Streptavidin-Dylight633-labeled thrombosomes in the presence of human plasma fibrinogen, in the GFP fluorescence channel.

FIG. 8B is an exemplary fluorescence microscopy image of a mixture of BODIPY-labeled PANSORBIN® and Streptavidin-Dylight633-labeled thrombosomes in the presence of human plasma fibrinogen, in the TexasRed fluorescence channel.

FIG. 8C is an exemplary fluorescence microscopy overlay image of a mixture of BODIPY-labeled PANSORBIN® and Streptavidin-Dylight633-labeled thrombosomes in the presence of human plasma fibrinogen, showing both the GFP and TexasRed fluorescence channels.

### DETAILED DESCRIPTION

It is to be understood that the terminology used herein is for the purpose of describing particular embodiments only, and is not intended to be limiting. Further, where a range of values is disclosed, the skilled artisan will understand that all other specific values within the disclosed range are inherently disclosed by these values and the ranges they represent without the need to disclose each specific value or range herein. For example, a disclosed range of 1-10 includes 1-9,1-5, 2-10, 3.1-6, 1, 2, 3, 4, 5, and so forth. In addition, each disclosed range includes up to 5% lower for the lower value of the range and up to 5% higher for the higher value of the range. For example, a disclosed range of 4-10 includes 3.8-10.5. This concept is captured in this document by the term “about”.

Unless defined otherwise, all technical and scientific terms used herein have the same meaning as commonly understood by one of ordinary skill in the art to which the term belongs. Although any methods and materials similar or equivalent to those described herein can be used in the practice or testing of the present disclosure, the preferred methods and materials are now described. All publications mentioned herein are incorporated herein by reference to disclose and describe the methods and/or materials in connection with which the publications are cited. The present disclosure is controlling to the extent it conflicts with any incorporated publication.

As used herein and in the appended claims, the term “platelet” can include whole platelets, fragmented platelets, platelet derivatives, or thrombosomes. “Platelets” within the above definition may include, for example, platelets in whole blood, platelets in plasma, platelets in buffer optionally supplemented with select plasma proteins, cold stored platelets, dried platelets, cryopreserved platelets, thawed cryopreserved platelets, rehydrated dried platelets, rehydrated cryopreserved platelets, lyopreserved platelets, thawed lyopreserved platelets, or rehydrated lyopreserved platelets. “Platelets” may be “platelets” of mammals, such as of humans, or such as of non-human mammals.

As used herein and in the appended claims, the singular forms “a”, “an”, and “the” include plural referents unless the context clearly dictates otherwise. Thus, for example, reference to “a platelet” includes a plurality of such platelets. Furthermore, the use of terms that can be described using equivalent terms include the use of those equivalent terms. Thus, for example, the use of the term “subject” is to be understood to include the terms “patient”, “individual” and other terms used in the art to indicate one who is subject to a treatment.

As used herein, “thrombosomes” are platelet derivatives that have been treated with an incubating agent (e.g., any of the incubating agents described herein) and lyopreserved (such as freeze-dried). In some cases, thrombosomes can be prepared from pooled platelets. Thrombosomes can have a shelf life of 2-3 years in dry form at ambient temperature and can be rehydrated with sterile water within minutes for immediate infusion. One example of thrombosomes are THROMBOSOMES®, which are in clinical trials for the treatment of acute hemorrhage in thrombocytopenic patients.

In some embodiments, rehydrating the platelets comprises adding to the platelets an aqueous liquid. In some embodiments, the aqueous liquid is water. In some embodiments, the aqueous liquid is an aqueous solution. In some embodiments, the aqueous liquid is a saline solution. In some embodiments, the aqueous liquid is a suspension.

In some embodiments, the rehydrated platelets have coagulation factor levels showing all individual factors (e.g., Factors VII, VIII and IX) associated with blood clotting at 40 international units (IU) or greater.

In some embodiments, the dried platelets, such as freeze-dried platelets, have less than about 10%, such as less than about 8%, such as less than about 6%, such as less than about 4%, such as less than about 2%, such as less than about 0.5% crosslinking of platelet membranes via proteins and/or lipids present on the membranes. In some embodiments, the rehydrated platelets, have less than about 10%, such as less than about 8%, such as less than about 6%, such as less than about 4%, such as less than about 2%, such as less than about 0.5% crosslinking of platelet membranes via proteins and/or lipids present on the membranes.

In some embodiments, the platelets and the dried platelets, such as freeze-dried platelets, having a particle size (e.g., diameter, max dimension) of at least about 0.2 μm (e.g., at least about 0.3 μm, at least about 0.4 μm, at least about 0.5 μm, at least about 0.6 μm, at least about 0.7 μm, at least about 0.8 μm, at least about 0.9 μm, at least about 1.0 μm, at least about 1.2 μm, at least about 1.5 μm, at least about 2.0 μm, at least about 2.5 μm, or at least about 5.0 μm). In some embodiments, the particle size is less than about 5.0 μm (e.g., less than about 2.5 μm, less than about 2.0 μm, less than about 1.5 μm, less than about 1.0 μm, less than about 0.9 μm, less than about 0.8 μm, less than about 0.7 μm, less than about 0.6 μm, less than about 0.5 μm, less than about 0.4 μm, or less than about 0.3 μm). In some embodiments, the particle size is from about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 um to about 0.9 μm, or from about 0.6 μm to about 0.8 μm).

In some embodiments, at least 50% (e.g., at least about 55%, at least about 60%, at least about 65%, at least about 70%, at least about 75%, at least about 80%, at least about 85%, at least about 90%, at least about 95%, or at least about 99%) of platelets and/or the dried platelets, such as freeze-dried platelets, have a particle size (e.g., in the largest dimension) in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, at most 99% (e.g., at most about 95%, at most about 80%, at most about 75%, at most about 70%, at most about 65%, at most about 60%, at most about 55%, or at most about 50%) of platelets and/or the dried platelets, such as freeze-dried platelets, are in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, about 50% to about 99% (e.g., about 55% to about 95%, about 60% to about 90%, about 65% to about 85, about 70% to about 80%) of platelets and/or the dried platelets, such as freeze-dried platelets, are in the range (e.g., in the largest dimension) of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some embodiments, platelets are isolated, for example in a liquid medium, prior to treating the disease or condition disclosed herein.

In some embodiments, platelets are donor-derived platelets. In some embodiments, platelets are obtained by a process that comprises an apheresis step. In some embodiments, platelets are pooled platelets.

In some embodiments, platelets such as lyophilized platelets, platelet derivatives, or thrombosomes are pooled from a plurality of donors. Such platelets, such as lyophilized platelets, platelet derivatives, and thrombosomes pooled from a plurality of donors may be also referred herein to as pooled platelets such as lyophilized platelets, platelet derivatives, or thrombosomes. In some embodiments, the donors are more than 5, such as more than 10, such as more than 20, such as more than 50, such as up to about 100 donors. In some embodiments, the donors are from about 5 to about 100, such as from about 10 to about 50, such as from about 20 to about 40, such as from about 25 to about 35.

In some embodiments, platelets are derived in vitro. In some embodiments, platelets are derived or prepared in a culture. In some embodiments, preparing the platelets comprises deriving or growing the platelets from a culture of megakaryocytes. In some embodiments, preparing the platelets comprises deriving or growing the platelets (or megakaryocytes) from a culture of human pluripotent stem cells (PCSs), including embryonic stem cells (ESCs) and/or induced pluripotent stem cells (iPSCs).

Accordingly, in some embodiments, platelets are prepared prior to treating the disease or condition disclosed herein. In some embodiments the platelets are lyophilized. In some embodiments the platelets are cryopreserved.

In some embodiments, the platelets or pooled platelets may be acidified to a pH of about 6.0 to about 7.4 prior to the incubation with the incubating agent. In some embodiments, the method comprises acidifying the platelets to a pH of about 6.5 to about 6.9. In some embodiments, the method comprises acidifying the platelets to a pH of about 6.6 to about 6.8. In some embodiments, the acidifying comprises adding to the pooled platelets a solution comprising Acid Citrate Dextrose (ACD).

In some embodiments, the platelets are isolated prior to the incubation with the incubating agent. In some embodiments, the method further comprises isolating platelets by using centrifugation. In some embodiments, the centrifugation occurs at a relative centrifugal force (RCF) of about 1000 ×g to about 2000 ×g. In some embodiments, the centrifugation occurs at relative centrifugal force (RCF) of about 1300 ×g to about 1800 ×g. In some embodiments, the centrifugation occurs at relative centrifugal force (RCF) of about 1500 ×g. In some embodiments, the centrifugation occurs for about 1 minute to about 60 minutes. In some embodiments, the centrifugation occurs for about 10 minutes to about 30 minutes. In some embodiments, the centrifugation occurs for about 30 minutes.

An incubating agent can include any appropriate components. In some embodiments, the incubating agent may comprise a liquid medium. In some embodiments the incubating agent may comprise one or more salts selected from phosphate salts, sodium salts, potassium salts, calcium salts, magnesium salts, and any other salt that can be found in blood or blood products, or that is known to be useful in drying platelets, or any combination of two or more of these.

In some embodiments, the incubating agent comprises one or more salts, such as phosphate salts, sodium salts, potassium salts, calcium salts, magnesium salts, and any other salt that can be found in blood or blood products. Exemplary salts include sodium chloride (NaCl), potassium chloride (KCl), and combinations thereof. In some embodiments, the incubating agent includes from about 0.5 mM to about 100 mM of the one or more salts. In some embodiments, the incubating agent includes from about 1 mM to about 100 mM (e.g., about 2 mM to about 90 mM, about 2 mM to about 6 mM, about 50 mM to about 100 mM, about 60 mM to about 90 mM, about 70 to about 85 mM) about of the one or more salts. In some embodiments, the incubating agent includes about 5 mM, about 75 mM, or about 80 mM of the one or more salts.

Preferably, these salts are present in the composition comprising platelets or platelet derivatives, such as freeze-dried platelets, at an amount that is about the same as is found in whole blood.

The incubating agent may be any buffer that is non-toxic to the platelets and provides adequate buffering capacity to the solution at the temperatures at which the solution will be exposed during the process provided herein. Thus, the buffer may comprise any of the known biologically compatible buffers available commercially, such as phosphate buffers, such as phosphate buffered saline (PBS), bicarbonate/carbonic acid, such as sodium-bicarbonate buffer, N-2-hydroxyethylpiperazine-N-2- ethanesulfonic acid (HEPES), and tris-based buffers, such as tris-buffered saline (TBS). Likewise, it may comprise one or more of the following buffers: propane- 1,2,3-tricarboxylic (tricarballylic); benzenepentacarboxylic; maleic; 2,2- dimethylsuccinic; EDTA; 3,3-dimethylglutaric; bis(2-hydroxyethyl)imino- tris (hydroxymethyl)-methane (BIS-TRIS); benzenehexacarboxylic (mellitic); N-(2- acetamido)imino-diacetic acid (ADA); butane-1,2,3,4-tetracarboxylic; pyrophosphoric; 1,1-cyclopentanediacetic (3,3 tetramethylene-glutaric acid); piperazine-1,4-bis -(2-ethanesulfonic acid) (PIPES); N-(2-acetamido)-2- amnoethanesulfonic acid (ACES); 1,1-cyclohexanediacetic; 3,6-endomethylene- 1,2,3,6-tetrahydrophthalic acid (EMTA; ENDCA); imidazole;; 2- (aminoethyl)trimethylammonium chloride (CHOLAMINE); N,N-bis (2- hydroxyethyl)-2-aminoethanesulfonic acid (BES); 2-methylpropane-1,2,3-triscarboxylic (beta-methyltricarballylic); 2-(N-morpholino)propane-sulfonic acid (MOPS); phosphoric; and N-tris(hydroxymethyl)methyl-2-amminoethane sulfonic acid (TES). In some embodiments, the incubating agent includes one or more buffers, e.g., N-2-hydroxyethylpiperazine -N′-2- ethanesulfonic acid (HEPES), or sodium-bicarbonate (NaHCO₃). In some embodiments, the incubating agent includes from about 5 to about 100 mM of the one or more buffers. In some embodiments, the incubating agent includes from about 5 to about 50 mM (e.g., from about 5 mM to about 40 mM, from about 8 mM to about 30 mM, about 10 mM to about 25 mM) about of the one or more buffers. In some embodiments, the incubating agent includes about 10 mM, about 20 mM, about 25 mM, or about 30 mM of the one or more buffers.

In some embodiments, the incubating agent includes one or more saccharides, such as monosaccharides and disaccharides, including sucrose, maltose, trehalose, glucose, mannose, dextrose, and xylose. In some embodiments, the saccharide includes a monosaccharide. In some embodiments, the saccharide includes a disaccharide. In some embodiments, the saccharide is a non-reducing disaccharide. In some embodiments, the saccharide is sucrose, maltose, trehalose, glucose (e.g., dextrose), mannose, or xylose. In some embodiments, the incubating agent comprises a starch. In some embodiments, the incubating agent includes polysucrose, a polymer of sucrose and epichlorohydrin. In some embodiments, the incubating agent includes from about 10 mM to about 1,000 mM of the one or more saccharides. In some embodiments, the incubating agent includes from about 50 mM to about 500 mM of the one or more saccharides. In embodiments, one or more saccharides is present in an amount of from 10 mM to 500 mM. In some embodiments, one or more saccharides is present in an amount of from 50 mM to 200 mM. In embodiments, one or more saccharides is present in an amount from 100 mM to 150 mM.

In some embodiments, the medium of the incubating agent may include human plasma, human whole blood, and/or an aqueous buffer (e.g., any of the buffers described herein). A buffer may be supplemented with appropriate concentrations of human plasma fibrinogen, Ca²⁺, and/or Mg²⁺. In some embodiments, the incubating agent includes approximately 1 mg/mL human plasma fibrinogen, approximately 1 mM Ca²⁺, and/or approximately 1 mM Mg²⁺.

In some embodiments, the compositions herein can comprise human platelets and a buffer comprising: approximately 9.5 mM HEPES, approximately 145 mM NaCl approximately 4.8 mM KCl, approximately 12 mM NaHCO₃, and approximately 0.35% BSA. The buffer may further comprise: approximately 1 mg/mL human plasma fibrinogen, approximately 1 mM Ca²⁺, approximately 1 mM Mg²⁺, and optionally human plasma and/or human whole blood.

In some embodiments, the compositions herein can comprise thrombosomes and a buffer comprising: approximately 9.5 mM HEPES, approximately 145 mM NaCl approximately 4.8 mM KCl, approximately 12 mM NaHCO₃, and approximately 0.35% BSA. The buffer may further comprise: approximately 1 mg/mL human plasma fibrinogen, approximately 1 mM Ca²⁺, approximately 1 mM Mg²⁺, and optionally human plasma and/or human whole blood.

In some embodiments, the compositions herein can comprise lyophilized platelets and a buffer comprising: approximately 9.5 mM HEPES, approximately 145 mM NaCl, approximately 4.8 mM KCl, approximately 12 mM NaHCO₃, and approximately 0.35% BSA. The buffer may further comprise: approximately 1 mg/mL human plasma fibrinogen, approximately 1 mM Ca²⁺, approximately 1 mM Mg²⁺, and optionally human plasma and/or human whole blood.

In some embodiments the composition comprising platelets or platelet derivatives, such as freeze-dried platelets, may comprise one or more of water or a saline solution. In some embodiments the composition comprising platelets or platelet derivatives, such as freeze-dried platelets, may comprise DMSO.

In some embodiments, the incubating agent comprises an organic solvent, such as an alcohol (e.g., ethanol). In such an incubating agent, the amount of solvent can range from 0.1% to 5.0% (v/v). In some embodiments, the organic solvent can range from about 0.1% (v/v) to about 5.0% (v/v), such as from about 0.3% (v/v) to about 3.0% (v/v), or from about 0.5% (v/v) to about 2% (v/v).

In some embodiments, suitable organic solvents include, but are not limited to alcohols, esters, ketones, ethers, halogenated solvents, hydrocarbons, nitriles, glycols, alkyl nitrates, water or mixtures thereof. In some embodiments, suitable organic solvents includes, but are not limited to methanol, ethanol, n-propanol, isopropanol, acetic acid, acetone, methyl ethyl ketone, methyl isobutyl ketone, methyl acetate, ethyl acetate, isopropyl acetate, tetrahydrofuran, isopropyl ether (IPE), tert-butyl methyl ether, dioxane (e.g., 1,4-dioxane), acetonitrile, propionitrile, methylene chloride, chloroform, toluene, anisole, cyclohexane, hexane, heptane, ethylene glycol, nitromethane, dimethylformamide, dimethyl sulfoxide, N-methyl pyrrolidone, dimethylacetamide, and combinations thereof. In some embodiments the organic solvent is selected from the group consisting of ethanol, acetic acid, acetone, acetonitrile, dimethylformamide, dimethyl sulfoxide (DMSO), dioxane, methanol, n-propanol, isopropanol, tetrahydrofuran (THF), N-methyl pyrrolidone, dimethylacetamide (DMAC), or combinations thereof. The presence of organic solvents, such as ethanol, can be beneficial in the processing of platelets such as lyophilized platelets, platelet derivatives, or thrombosomes (e.g., freeze-dried platelet derivatives).

In some embodiments the incubating agent is incubated into the platelets in the presence of an aqueous medium. In some embodiments the incubating agent is incubated in the presence of a medium comprising DMSO.

In some embodiments, one or more other components may be incubated in the platelets. Exemplary components may include Prostaglandin El or Prostacyclin and or EDTA/EGTA to prevent platelet aggregation and activation during the incubating process.

Non-limiting examples of incubating agent compositions that may be used are shown in Tables 1-5.

TABLE 1
Buffer

| | Concentration |
|---|---|
| Component | (mM unless otherwise specified) |
| NaCl | 75.0 |
| KCl | 4.8 |
| HEPES | 9.5 |
| NaHCO3 | 12.0 |
| Dextrose | 3 |
| Trehalose | 100 |
| Ethanol | 1% (v/v) |

Table 1. An incubating agent that can be used (e.g., to load platelets via endocytosis at 37° C. with gentle agitation as sample is placed on a rocker. Adjust pH to 6.6-6.8)

TABLE 2
Buffer A

| | Concentration |
|---|---|
| Component | (mM unless specified otherwise) |
| CaCl₂ | 1.8 |
| MgCl₂ | 1.1 |
| KCl | 2.7 |
| NaCl | 137 |
| NaH₂PO₄ | 0.4 |
| HEPES | 10 |
| D-glucose | 5.6 |
| pH | 6.5 |

Table 2. An incubating agent that can be used. Exemplary incubation is performed done at 37° C. with gentle agitation as sample is placed on a rocker.

TABLE 3
Buffer B

| | Concentration |
|---|---|
| Component | (mM unless otherwise specified) |
| Buffer and Salts | Table 4 (below) |
| BSA | 0.35% |
| Dextrose | 5 |
| pH | 7.4 |

Table 3. Buffer B can used when incubating platelets, e.g., for flow cytometry. Such an incubation can be done at room temperature in the dark. Albumin is an optional component of Buffer B.

TABLE 4
Concentration of HEPES and of Salts in Buffer B

| | Concentration |
|---|---|
| Component | (mM unless otherwise specified) |
| HEPES | 25 |
| NaCl | 119 |
| KCl | 5 |
| CaCl₂ | 2 |
| MgCl₂ | 2 |
| glucose | 6 g/l |

Table 4 is another exemplary incubating agent. The pH can be adjusted to 7.4 with NaOH. Albumin is an optional component of Buffer B.

TABLE 5
Tyrode's HEPES Buffer (plus PGE1)

| Component | Concentration (mM) |
|---|---|
| CaCl₂ | 1.8 |
| MgCl₂ | 1.1 |
| KCl | 2.7 |
| NaCl | 137 |
| NaH₂PO₄ | 0.4 |
| HEPES | 10 |
| D-glucose | 5.6 |
| pH | 6.5 |
| Prostagalandin E1 (PGE1) | 1 μg/ml |

Table 5 is another exemplary incubating agent.

In some embodiments, the platelets are incubated with the incubating agent for different durations at or at different temperatures from 15-45° C., or about 37° C.

In some embodiments, the platelets form a suspension in an incubating agent comprising a liquid medium at a concentration from 10,000 platelets/μL to 10,000,000 platelets/μL, such as 50,000 platelets/μL to 2,000,000 platelets/μL, such as 100,000 platelets/μL to 500,000 platelets/μL, such as 150,000 platelets/μL to 300,000 platelets/μL, such as 200,000 platelets/μL.

The platelets may be incubated with the incubating agent for different durations, such as, for example, for at least about 5 minutes (mins) (e.g., at least about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, about 42 hrs, about 48 hrs, or at least about 48 hrs. In some embodiments, the platelets may be incubated with the incubating agent for no more than about 48 hrs (e.g., no more than about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, or no more than about 42 hrs). In some embodiments, the platelets may be incubated with the incubating agent for from about 10 mins to about 48 hours (e.g., from about 20 mins to about 36 hrs, from about 30 mins to about 24 hrs, from about 1 hr to about 20 hrs, from about 2 hrs to about 16 hours, from about 10 mins to about 24 hours, from about 20 mins to about 12 hours, from about 30 mins to about 10 hrs, or from about 1 hr to about 6 hrs. In some embodiments, the platelets such as lyophilized platelets, the platelet derivatives, or the thrombosomes are incubated with the incubating agent for a period of time of 5 minutes to 48 hours, such as 10 minutes to 24 hours, such as 20 minutes to 12 hours, such as 30 minutes to 6 hours, such as 1 hour minutes to 3 hours, such as about 2 hours.

In some embodiments, the platelets are incubated with the incubating agents at different temperatures. In embodiments, incubation is conducted at 37° C. In certain embodiments, incubation is performed at 4° C. to 45° C., such as 15° C. to 42° C. For example, in embodiments, incubation is performed at 35° C. to 40° C. (e.g., 37° C.) for 110 to 130 (e.g., 120) minutes and for as long as 24-48 hours. In some embodiments, the platelets are incubated with the incubating agent for different durations as disclosed herein, and at temperatures from 15-45° C., or about 37° C.

In some embodiments, the method further comprises drying the platelets. In some embodiments, the drying step comprises freeze-drying the platelets. In some embodiments, the method further comprises rehydrating the platelets obtained from the drying step.

In some embodiments, the platelets are cold stored, cryopreserved, or lyophilized (in some embodiments, in the production of thrombosomes) prior to use in therapy or in functional assays.

Any known technique for drying platelets can be used in accordance with the present disclosure, as long as the technique can achieve a final residual moisture content of less than 5%. Preferably, the technique achieves a final residual moisture content of less than 2%, such as 1%, 0.5%, or 0.1%. Non-limiting examples of suitable techniques are freeze-drying (lyophilization) and spray-drying. A suitable lyophilization method is presented in Table A. Additional exemplary lyophilization methods can be found in U.S. Pat. Nos. 7,811,558, 8,486,617, and U.S. Pat. No. 8,097,403. An exemplary spray-drying method includes: combining nitrogen, as a drying gas, with a incubating agent according to the present disclosure, then introducing the mixture into GEA Mobile Minor spray dryer from GEA Processing Engineering, Inc. (Columbia Md. USA), which has a Two-Fluid Nozzle configuration, spray drying the mixture at an inlet temperature in the range of 150° C. to 190° C., an outlet temperature in the range of 65° C. to 100° C., an atomic rate in the range of 0.5 to 2.0 bars, an atomic rate in the range of 5 to 13 kg/hr, a nitrogen use in the range of 60 to 100 kg/hr, and a run time of10 to 35 minutes. The final step in spray drying is preferentially collecting the dried mixture. The dried composition in some embodiments is stable for at least six months at temperatures that range from −20° C. or lower to 90° C. or higher.

TABLE A
Exemplary Lyophilization Protocol

| | | | | | | | Pressure |
|---|---|---|---|---|---|---|---|
| | Step | Temp. Set | | Type | Duration | | Set |
| Freezing Step | F1 | −50° | C. | Ramp | Var | | N/A |
| | F2 | −50° | C. | Hold | 3 | Hrs | N/A |
| Vacuum Pulldown | F3 | −50° | | Hold | Var | | N/A |
| Primary Dry | P1 | −40° | | Hold | 1.5 | Hrs | 0 mT |
| | P2 | −35° | | Ramp | 2 | Hrs | 0 mT |
| | P3 | −25° | | Ramp | 2 | Hrs | 0 mT |
| | P4 | −17° | C. | Ramp | 2 | Hrs | 0 mT |
| | P5 | 0° | C. | Ramp | 1.5 | Hrs | 0 mT |
| | P6 | 27° | C. | Ramp | 1.5 | Hrs | 0 mT |
| | P7 | 27° | C. | Hold | 16 | Hrs | 0 mT |
| Secondary Dry | S1 | 27° | C. | Hold | >8 | Hrs | 0 mT |

In some embodiments, the step of drying the platelets that are obtained as disclosed herein, such as the step of freeze-drying the platelets that are obtained as disclosed herein, comprises incubating the platelets with a lyophilizing agent (e.g., a non-reducing disaccharide). Accordingly, in some embodiments, the methods for preparing platelets further comprise incubating the platelets with a lyophilizing agent. In some embodiments the lyophilizing agent is a saccharide. In some embodiments the saccharide is a disaccharide, such as a non-reducing disaccharide.

In some embodiments, the platelets are incubated with a lyophilizing agent for a sufficient amount of time and at a suitable temperature to incubate the platelets with the lyophilizing agent. Non-limiting examples of suitable lyophilizing agents are saccharides, such as monosaccharides and disaccharides, including sucrose, maltose, trehalose, glucose (e.g., dextrose), mannose, and xylose. In some embodiments, non-limiting examples of lyophilizing agent include serum albumin, dextran, polyvinyl pyrolidone (PVP), starch, and hydroxyethyl starch (HES). In some embodiments, exemplary lyophilizing agents can include a high molecular weight polymer. By “high molecular weight” it is meant a polymer having an average molecular weight of about or above 70 kDa and up to 1,000,000 kDa. Non-limiting examples are polymers of sucrose and epichlorohydrin (e.g., poly sucrose). In some embodiments, the lyophilizing agent is polysucrose. Although any amount of high molecular weight polymer can be used as a lyophilizing agent, it is preferred that an amount be used that achieves a final concentration of about 3% to 10% (w/v), such as 3% to 7%, for example 6%.

An exemplary saccharide for use in the compositions disclosed herein is trehalose. Regardless of the identity of the saccharide, it can be present in the composition in any suitable amount. For example, it can be present in an amount of 1 mM to 1 M. In embodiments, it is present in an amount of from 10 mM 10 to 500 mM. In some embodiments, it is present in an amount of from 20 mM to 200 mM. In embodiments, it is present in an amount from 40 mM to 100 mM. In various embodiments, the saccharide is present in different specific concentrations within the ranges recited above, and one of skill in the art can immediately understand the various concentrations without the need to specifically recite each herein. Where more than one saccharide is present in the composition, each saccharide can be present in an amount according to the ranges and particular concentrations recited above.

Within the process provided herein for making the compositions provided herein, addition of the lyophilizing agent can be the last step prior to drying. However, in some embodiments, the lyophilizing agent is added at the same time or before other components of the composition, such as a salt, a buffer, optionally a cryoprotectant, or other components. In some embodiments, the lyophilizing agent is added to the incubating agent, thoroughly mixed to form a drying solution, dispensed into a drying vessel (e.g., a glass or plastic serum vial, a lyophilization bag), and subjected to conditions that allow for drying of the solution to form a dried composition.

The step of incubating the platelets with a cryoprotectant includes incubating the platelets for a time suitable for loading, as long as the time, taken in conjunction with the temperature, is sufficient for the cryoprotectant to come into contact with the platelets and, preferably, be incorporated, at least to some extent, into the platelets. In embodiments, incubation is carried out for about 1 minute to about 180 minutes or longer.

The step of incubating the platelets with a cryoprotectant includes incubating the platelets and the cryoprotectant at a temperature that, when selected in conjunction with the amount of time allotted, is suitable for incubating. In general, the composition is incubated at a temperature above freezing for at least a sufficient time for the cryoprotectant to come into contact with the platelets. In embodiments, incubation is conducted at 37° C. In certain embodiments, incubation is performed at 20° C. to 42° C. For example, in embodiments, incubation is performed at 35° C. to 40° C. (e.g., 37° C.) for 110 to 130 (e.g., 120) minutes.

In various embodiments, the lyophilization bag is a gas-permeable bag configured to allow gases to pass through at least a portion or all portions of the bag during the processing. The gas-permeable bag can allow for the exchange of gas within the interior of the bag with atmospheric gas present in the surrounding environment. The gas-permeable bag can be permeable to gases, such as oxygen, nitrogen, water, air, hydrogen, and carbon dioxide, allowing gas exchange to occur in the compositions provided herein. In some embodiments, the gas-permeable bag allows for the removal of some of the carbon dioxide present within an interior of the bag by allowing the carbon dioxide to permeate through its wall. In some embodiments, the release of carbon dioxide from the bag can be advantageous to maintaining a desired pH level of the composition contained within the bag.

In some embodiments, the container of the process herein is a gas-permeable container that is closed or sealed. In some embodiments, the container is a container that is closed or sealed and a portion of which is gas-permeable. In some embodiments, the surface area of a gas-permeable portion of a closed or sealed container (e.g., bag) relative to the volume of the product being contained in the container (hereinafter referred to as the “SA/V ratio”) can be adjusted to improve pH maintenance of the compositions provided herein. For example, in some embodiments, the SA/V ratio of the container can be at least about 2.0 cm²/mL (e.g., at least about 2.1 cm²/mL, at least about 2.2 cm²/mL, at least about 2.3 cm²/mL, at least about 2.4 cm²/mL, at least about 2.5 cm²/mL, at least about 2.6 cm²/mL, at least about 2.7 cm²/mL, at least about 2.8 cm²/mL, at least about 2.9 cm²/mL, at least about 3.0 cm²/mL, at least about 3.1 cm²/mL, at least about 3.2 cm²/mL, at least about 3.3 cm²/mL, at least about 3.4 cm²/mL, at least about 3.5 cm²/mL, at least about 3.6 cm²/mL, at least about 3.7 cm²/mL, at least about 3.8 cm²/mL, at least about 3.9 cm²/mL, at least about 4.0 cm²/mL, at least about 4.1 cm²/mL, at least about 4.2 cm²/mL, at least about 4.3 cm²/mL, at least about 4.4 cm²/mL, at least about 4.5 cm²/mL, at least about 4.6 cm²/mL, at least about 4.7 cm²/mL, at least about 4.8 cm²/mL, at least about 4.9 cm²/mL, or at least about 5.0 cm²/mL. In some embodiments, the SA/V ratio of the container can be at most about 10.0 cm²/mL (e.g., at most about 9.9 cm²/mL, at most about 9.8 cm²/mL, at most about 9.7 cm²/mL, at most about 9.6 cm²/mL, at most about 9.5 cm²/mL, at most about 9.4 cm²/mL, at most about 9.3 cm²/mL, at most about 9.2 cm²/mL, at most about 9.1 cm²/mL, at most about 9.0 cm²/mL, at most about 8.9 cm²/mL, at most about 8.8 cm²/mL, at most about 8.7 cm²/mL, at most about 8.6, cm²/mL at most about 8.5 cm²/mL, at most about 8.4 cm²/mL, at most about 8.3 cm²/mL, at most about 8.2 cm²/mL, at most about 8.1 cm²/mL, at most about 8.0 cm²/mL, at most about 7.9 cm²/mL, at most about 7.8 cm²/mL, at most about 7.7 cm²/mL, at most about 7.6 cm²/mL, at most about 7.5 cm²/mL, at most about 7.4 cm²/mL, at most about 7.3 cm²/mL, at most about 7.2 cm²/mL, at most about 7.1 cm²/mL, at most about 6.9 cm²/mL, at most about 6.8 cm²/mL, at most about 6.7 cm²/mL, at most about 6.6 cm²/mL, at most about 6.5 cm²/mL, at most about 6.4 cm²/mL, at most about 6.3 cm²/mL, at most about 6.2 cm²/mL, at most about 6.1 cm²/mL, at most about 6.0 cm²/mL, at most about 5.9 cm²/mL, at most about 5.8 cm²/mL, at most about 5.7 cm²/mL, at most about 5.6 cm²/mL, at most about 5.5 cm²/mL, at most about 5.4 cm²/mL, at most about 5.3 cm²/mL, at most about 5.2 cm²/mL, at most about 5.1 cm²/mL, at most about 5.0 cm²/mL, at most about 4.9 cm²/mL, at most about 4.8 cm²/mL, at most about 4.7 cm²/mL, at most about 4.6 cm²/mL, at most about 4.5 cm²/mL, at most about 4.4 cm²/mL, at most about 4.3 cm²/mL, at most about 4.2 cm²/mL, at most about 4.1 cm²/mL, or at most about 4.0 cm²/mL. In some embodiments, the SA/V ratio of the container can range from about 2.0 to about 10.0 cm²/mL (e.g., from about 2.1 cm²/mL to about 9.9 cm²/mL, from about 2.2 cm²/mL to about 9.8 cm²/mL, from about 2.3 cm²/mL to about 9.7 cm²/mL, from about 2.4 cm²/mL to about 9.6 cm²/mL, from about 2.5 cm²/mL to about 9.5 cm²/mL, from about 2.6 cm²/mL to about 9.4 cm²/mL, from about 2.7 cm²/mL to about 9.3 cm²/mL, from about 2.8 cm²/mL to about 9.2 cm²/mL, from about 2.9 cm²/mL to about 9.1 cm²/mL, from about 3.0 cm²/mL to about 9.0 cm²/mL, from about 3.1 cm²/mL to about 8.9 cm²/mL, from about 3.2 cm²/mL to about 8.8 cm²/mL, from about 3.3 cm²/mL to about 8.7 cm²/mL, from about 3.4 cm²/mL to about 8.6 cm²/mL, from about 3.5 cm²/mL to about 8.5 cm²/mL, from about 3.6 cm²/mL to about 8.4 cm²/mL, from about 3.7 cm²/mL to about 8.3 cm²/mL, from about 3.8 cm²/mL to about 8.2 cm²/mL, from about 3.9 cm²/mL to about 8.1 cm²/mL, from about 4.0 cm²/mL to about 8.0 cm²/mL, from about 4.1 cm²/mL to about 7.9 cm²/mL, from about 4.2 cm²/mL to about 7.8 cm²/mL, from about 4.3 cm²/mL to about 7.7 cm²/mL, from about 4.4 cm²/mL to about 7.6 cm²/mL, from about 4.5 cm²/mL to about 7.5 cm²/mL, from about 4.6 cm²/mL to about 7.4 cm²/mL, from about 4.7 cm²/mL to about 7.3 cm²/mL, from about 4.8 cm²/mL to about 7.2 cm²/mL, from about 4.9 cm²/mL to about 7.1 cm²/mL, from about 5.0 cm²/mL to about 6.9 cm²/mL, from about 5.1 cm²/mL to about 6.8 cm²/mL, from about 5.2 cm²/mL to about 6.7 cm²/mL, from about 5.3 cm²/mL to about 6.6 cm²/mL, from about 5.4 cm²/mL to about 6.5 cm²/mL, from about 5.5 cm²/mL to about 6.4 cm²/mL, from about 5.6 cm²/mL to about 6.3 cm²/mL, from about 5.7 cm²/mL to about 6.2 cm²/mL, or from about 5.8 cm²/mL to about 6.1 cm²/mL.

Gas-permeable closed containers (e.g., bags) or portions thereof can be made of one or more various gas-permeable materials. In some embodiments, the gas-permeable bag can be made of one or more polymers including fluoropolymers (such as polytetrafluoroethylene (PTFE) and perfluoroalkoxy (PFA) polymers), polyolefins (such as low-density polyethylene (LDPE), high-density polyethylene (HDPE)), fluorinated ethylene propylene (FEP), polystyrene, polyvinylchloride (PVC), silicone, and any combinations thereof.

In some embodiments, dried platelets, such as lyophilized platelets, or platelet derivatives (e.g., thrombosomes) can undergo heat treatment. Heating can be performed at a temperature above about 25° C. (e.g., greater than about 40° C., 50° C., 60° C., 70° C., 80° C. or higher). In some embodiments, heating is conducted between about 70° C. and about 85° C. (e.g., between about 75° C. and about 85° C., or at about 75° C. or 80° C.). The temperature for heating can be selected in conjunction with the length of time that heating is to be performed. Although any suitable time can be used, typically, the lyophilized platelets are heated for at least 1 hour, but not more than 36 hours. Thus, in embodiments, heating is performed for at least 2 hours, at least 6 hours, at least 12 hours, at least 18 hours, at least 20 hours, at least 24 hours, or at least 30 hours. For example, the lyophilized platelets can be heated for 18 hours, 19 hours, 20 hours, 21 hours, 22 hours, 23 hours, 24 hours, 25 hours, 26 hours, 27 hours, 28 hours, 29 hours, or 30 hours. Non-limiting exemplary combinations include: heating the dried platelets, such as lyophilized platelets, or platelet derivatives (e.g., thrombosomes) for at least 30 minutes at a temperature higher than 30° C.; heating the dried platelets, such as lyophilized platelets, or platelet derivatives (e.g., thrombosomes) for at least 10 hours at a temperature higher than 50° C.; heating the dried platelets, such as lyophilized platelets, or platelet derivatives (e.g., thrombosomes) for at least 18 hours at a temperature higher than 75° C.; and heating the dried platelets, such as lyophilized platelets, or platelet derivatives (e.g., thrombosomes) for 24 hours at 80° C. In some embodiments, heating can be performed in sealed container, such as a capped vial. In some embodiments, a sealed container can be subjected to a vacuum prior to heating. The heat treatment step, particularly in the presence of a cryoprotectant such as albumin or polysucrose, has been found to improve the stability and shelf-life of the freeze-dried platelets. Indeed, advantageous results have been obtained with the particular combination of serum albumin or polysucrose and a post-lyophilization heat treatment step, as compared to those cryoprotectants without a heat treatment step. A cryoprotectant (e.g., sucrose) can be present in any appropriate amount (e.g. about 3% to about 10% by mass or by volume of the platelets, such as lyophilized platelets, or platelet derivatives (e.g., thrombosomes).

In some embodiments, the platelets prepared as disclosed herein by a process comprising incubation with an incubating agent have a storage stability that is at least about equal to that of the platelets prior to the incubation.

In some embodiments, the method further comprises cryopreserving the platelets, or platelet derivatives prior to administering the platelets or platelet derivatives (e.g., with an incubating agent, e.g., an incubating agent described herein).

In some embodiments, the method further comprises drying a composition comprising platelets or platelet derivatives, (e.g., with an incubating agent e.g., an incubating agent described herein) prior to administering the platelets such as lyophilized platelets, platelet derivatives, or thrombosomes. In some embodiments, the method may further comprise heating the composition following the drying step. In some embodiments, the method may further comprise rehydrating the composition following the freeze-drying step or the heating step.

In some embodiments, the method further comprises freeze-drying a composition comprising platelets or platelet derivatives (e.g., with an incubating agent e.g., an incubating agent described herein) prior to administering the platelets such as lyophilized platelets, platelet derivatives, or thrombosomes. In some embodiments, the method may further comprise heating the composition following the freeze-drying step. In some embodiments, the method may further comprise rehydrating the composition following the freeze-drying step or the heating step.

In some embodiments, the method further comprises cold storing the platelets, such as lyophilized platelets, the platelet derivatives, or the thrombosomes prior to administering the platelets such as lyophilized platelets, platelet derivatives, or thrombosomes (e.g., with an incubating agent, e.g., an incubating agent described herein).

Storing conditions include, for example, standard room temperature storing (e.g., storing at a temperature ranging from about 20 to about 30° C.) or cold storing (e.g., storing at a temperature ranging from about 1 to about 10° C.). In some embodiments, the method further comprises cryopreserving, freeze-drying, thawing, rehydrating, and combinations thereof, a composition comprising platelets such as lyophilized platelets, platelet derivatives, or thrombosomes (e.g., with an incubating agent e.g., an incubating agent described herein) prior to administering the platelets such as lyophilized platelets, platelet derivatives, or thrombosomes. For example, in some embodiments, the method further comprises drying (e.g., freeze-drying) a composition comprising platelets or platelet derivatives (e.g., with an incubating agent e.g., an incubating agent described herein) (e.g., to form thrombosomes) prior to administering the platelets such as lyophilized platelets, platelet derivatives, or thrombosomes. In some embodiments, the method may further comprise rehydrating the composition obtained from the drying step.

In some cases, pathogenic bodies (e.g., bacteria, fungi, protozoa, viruses) can cause infection and/or sepsis. Current treatments for sepsis include: antibiotics and steroids to fight infection; supportive care to increase blood pressure and prevent dehydration. In cases of kidney failure, patients may need dialysis. There is no widely practiced clinical method for physically removing infectious particles from the circulation. Provided herein are such methods.

Without wishing to be bound by theory or mechanism, applicants believe that the platelets or platelet derivatives herein bind foreign bodies, such as pathogenic bodies, such as Staphylococcus aureus, a gram-positive coccus bacterium with human pathogenicity. The binding facilitates recruitment of an immune response and/or clearance from the circulation in vivo of such foreign bodies. The platelets or platelet derivatives have a short circulation lifetime and accumulate in the liver, where the foreign bodies bound to the platelets or platelet derivatives may be acted upon by innate and/or adaptive immune responses. The platelet derivatives herein may also help to recruit additional immune responses before and after this clearance. S. aureus and other bacteria expressing MSCRAMMs (microbial surface components recognizing adhesive matrix molecules) bind fibrinogen, among other matrix proteins, that facilitate interactions with platelets. Such MSCRAMMs include clumping factor A (ClfA), fibronectin binding protein A (FnbpA), and others.

S. aureus is associated with human pathogenicity with presentations that include sepsis and infective endocarditis. S. aureus has been demonstrated to bind and activate platelets in vitro and in vivo, but modeling these interactions in the laboratory environment requires careful handling of potentially pathogenic bacterial strains.

PANSORBIN® is a heat-killed, formalin fixed S. aureus of strain Cowan 1. The Cowan 1 strain of S. aureus used to make PANSORBIN® expresses surface proteins required for fibrinogen binding. Cowan 1 overexpresses Protein A. Protein A binds the Fc region of IgG, facilitating binding to antibodies for immunoprecipitation, detection of antibody bound to immobilized substrate, etc. This feature of S. aureus helps the bacteria evade immune cells due to competitive antibody binding, which prevents proper recognition of antigen-antibody interactions on the bacteria by the immune system. PANSORBIN® can be safely handled in the general laboratory environment and does not require cell culture capabilities or specialized storage conditions for extended study. The described bacteria are commercially sold under the name PANSORBIN® for use in antibody purification, mitogenic stimulation of B cells, and immunoprecipitation applications.

PANSORBIN® cells pass the slide coagulase assay by clumping in the presence of human plasma, indicating they bind to soluble fibrinogen immobilized on the surface of a glass slide. This suggests that S. aureus MSCRAMMs are present and functional on the surface of the fixed cells and are capable of modeling binding interactions that would occur with live cells.

Also provided herein are methods of treatment. For example, provided herein are methods of treating a subject with an infection, the method comprising administering to the subject in need thereof an effective amount of a composition comprising platelets such as lyophilized platelets, or platelet derivatives (e.g., thrombosomes, optionally rehydrated) and an incubating agent comprising one or more salts, a buffer, optionally a cryoprotectant, and optionally an organic solvent. Also provided herein are methods of treating an infection in a subject, the method comprising administering to the subject in need thereof an effective amount of a composition prepared by incubating platelets with an incubating agent comprising one or more salts, a buffer, optionally a cryoprotectant, and optionally an organic solvent. An incubating agent can be any appropriate incubating agent. An incubating agent can be any of the incubating agents described herein. In some embodiments, the process for preparing the composition comprising platelets and an incubating agent includes drying a composition comprising platelets and an incubating agent. Drying can include any appropriate steps. In some embodiments, drying includes freeze-drying. In some embodiments, the process for preparing the composition comprising platelets and an incubating agent includes rehydrating a composition comprising platelets and an incubating agent. A composition comprising platelets and an incubating agent can be rehydrated using any suitable method. In some embodiments, the composition can be rehydrated with water. In some embodiments, the composition can be rehydrated with a buffer. A buffer for rehydration can be any appropriate buffer, for example, any of the incubating agents described herein.

Provided herein in some embodiments is a method of treating a disease or condition as disclosed herein, wherein the method comprises pooling platelets such as lyophilized platelets, platelet derivatives, or thrombosomes from a plurality of donors, prior to administering the composition as disclosed herein.

Examples of diseases (therapeutic indications) that may be treated with the compositions disclosed herein are as follows. In some embodiments, a disease that may be treated with the compositions disclosed herein include vasculitis or a vascular leak (e.g., such as that brought on by endotheliopathy). In some embodiments, a disease that may be treated with the compositions disclosed herein can include an infection. An infection can be any appropriate infection. For example, the infection can be a bacterial infection, a fungal infection, a protozoan infection, or a viral infection. In some embodiments, the infection can be a bacterial infection (e.g., an antibiotic resistant bacterial infection). A bacterial infection can be any appropriate bacterial infection. In some embodiments, the bacterial infection can be a Staphylococcus aureus infection. In some embodiments, the bacterial infection can be a gram negative bacterial infection. A gram negative bacterial infection can be any appropriate gram negative bacterial infection. In some embodiments, the gram negative bacterial infection can be caused by E. coli, Pseudomonas aeruginosa, Neisseria gonorrhoeae, Chlamydia trachomatis, Yersinia pestis, or two or more thereof. In some embodiments, the bacterial infection can be a gram positive bacterial infection. A gram positive bacterial infection can be any appropriate gram positive bacterial infection. In some embodiments, the gram positive bacterial infection can be caused by Bacillus anthracis, Corynebacterium diphtherias, Enterococcus faecalis, Enterococcus faecium, Erysipelothrix rhusiopathiae, Listeria monocytogenes, Streptococcus pneumonia, Staphylococcus aureus, Streptococcus pyogenes, Streptococcus agalactiae, Viridans streptococci, or two or more thereof. In some embodiments, the infection can be a fungal infection. A fungal infection can be any appropriate fungal infection. In some embodiments, the fungal infection can be caused by an Aspergillus species, a Blastomyces species, a Candida species, a Coccidioides species, a Cryptococcus species, a Histoplasma species, a Pneumocystis species, or two or more thereof. In some embodiments, an infection can be a protozoan infection. A protozoan infection can be any appropriate protozoan infection. For example, in some embodiments, a protozoan infection can be caused by an Entamoeba species, a Plasmodium species, a Giardia species, a Trypanosoma species, a Leishmania species, a Toxoplasma species, or two or more thereof. In some embodiments, an infection can be a viral infection. A viral infection can be any appropriate viral infection. For example, in some embodiments, a viral infection can be caused by a member of one or more of the following families: Adenoviridae, Herpesviridae, Papillomaviridae, Polyomaviridae, Poxviridae, Hepadnaviridae, Parvoviridae, Astroviridae, Caliciviridae, Picornaviridae, Coronaviridae (e.g., Alphacoronavirus, Betacoronavirus, Deltacoronavirus, or Gammacoronavirus), Flaviviridae, Togaviridae, Hepeviridae, Retroviridae, Orthomyxoviridae, Arenaviridae, Bunyaviridae, Filoviridae, Paramyxoviridae, Rhabdoviridae, or Reoviridae. In some embodiments, a viral infection can be a Coronaviridae infection. In some embodiments, a viral infection can be a Betacoronavirus infection (e.g., SARS-CoV, MERS-CoV, or SARS-CoV-2). In some embodiments, the viral infection can be SARS-CoV-2. In some embodiments, the infection can be a hemorrhagic viral infection. A hemorrhagic viral infection can be any appropriate hemorrhagic viral infection. For example, in some embodiments, the hemorrhagic viral infection can be caused by is Ebola virus, Marburg Virus, Lassa virus, or Dengue virus. In some embodiments, the infection can be a non-hemorrhagic viral infection. A non-hemorrhagic viral infection can be any appropriate non-hemorrhagic viral infection. For example, in some embodiments, a non-hemorrhagic viral infection can be caused by a member of one or more of the following families: Adenoviridae, Herpesviridae, Papillomaviridae, Polyomaviridae, Poxviridae, Hepadnaviridae, Parvoviridae, Astroviridae, Caliciviridae, Picornaviridae, Coronaviridae, Flaviviridae, Togaviridae, Hepeviridae, Retroviridae, Orthomyxoviridae, Arenaviridae, Bunyaviridae, Paramyxoviridae, Rhabdoviridae, or Reoviridae. In some embodiments, a member of the Retroviridae family is a human immunodeficiency virus (HIV) (e.g., HIV-1 or HIV-2).

In some embodiments, one or more symptoms of the infection can decrease following administration of an effective amount of a composition comprising platelets such as lyophilized platelets, or platelet derivatives (e.g., thrombosomes, optionally rehydrated). Non-limiting examples of symptoms of an infection include fever, chills, sweats, coughing, sore throat, shortness of breath, nasal congestion, stiff neck, burning or pain with urination, unusual vaginal discharge or irritation, increased urination, redness, soreness, or swelling, diarrhea, vomiting, pain in the abdomen or rectum, or new onset of pain. Additional non-limiting examples of symptoms of an infection (e.g., sepsis) can include patches of discolored skin, decreased urination, changes in mental ability, confusion, disorientation, low platelet count, problems breathing, shortness of breath, high heart rate, abnormal heart function(s), fever, feelings of cold, chills (e.g., due to a decrease of body temperature), shivering, clammy or sweaty skin, discomfort, pain, or unconsciousness. In some embodiments, for example, when the infection is a hemorrhagic virus infection, symptoms can include fever, bleeding, and/or nausea. The amount of the one or more symptoms of the infection can decrease by any appropriate amount. In some cases, one or more symptoms of the infection can disappear. A decrease in a symptom of the infection can be measured by any appropriate method.

Also provided herein is a method of binding a foreign body in a subject, the method comprising administering to the subject in need thereof an effective amount of a composition comprising platelets such as lyophilized platelets, or platelet derivatives (e.g., thrombosomes, optionally rehydrated) and an aqueous medium comprising one or more salts, a buffer, optionally a cryoprotectant, and optionally an organic solvent, wherein the foreign body binds to the platelets such as lyophilized platelets, or platelet derivatives (e.g., thrombosomes). The foreign body can be any appropriate foreign body. For example, the foreign body can be a pathogen. A pathogen can be any appropriate pathogen. In some embodiments, the pathogen can be a bacterium (e.g., Staphylococcus aureus), a fungus, a protozoa, or a virus. A bacterium, a fungus, a protozoa, or a virus can be any appropriate bacterium, fungus, protozoa, or virus, for example, any of the bacteria, fungi, protozoa, or viruses described herein. The foreign body can be located in or on the body of the subject in any appropriate location. For example, in some embodiments, the foreign body can be in the bloodstream of the subject. In some embodiments, the foreign body can be on the skin of the subject. In some embodiments, the foreign body can be in a wound (e.g., a surgical wound or a non-surgical wound) of a subject.

In some embodiments, the amount of the foreign body in or on the subject can decrease following administration of an effective amount of a composition comprising platelets such as lyophilized platelets, or platelet derivatives (e.g., thrombosomes, optionally rehydrated). A decrease in the foreign body can be measured using any appropriate time points, for example, before treatment and after administration of an effective amount of a composition comprising platelets or platelet derivatives. The amount of the foreign body can decrease by any appropriate amount. In some embodiments, the amount of the foreign body can decrease to a level undetectable by an appropriate assay. A decrease in the foreign body can be measured by any appropriate method.

For example, a bacterial or viral nucleic acid (e.g., one or more parts of the genome) can be detected by nucleic acid amplification (e.g., RT-PCR) in blood specimens. As another example, the non-structural-1 (NS1) bacterial or viral antigens can be detected (e.g., up to day four post-onset) by any appropriate method (e.g., using an antibody specific to the particular NS1 antigen, e.g., in an enzyme-linked immunosorbent assay (ELISA)). As another example, viral or bacterial isolation may be performed to culture the infectious agent to determine the viral or bacterial titer. An increase of an antibody titre (e.g., dengue or other viral or bacterial IgG) can also be measured and used as a surrogate for a decrease in the amount of the foreign body. As another example, serological analysis can be performed by detection of, for example, a host antibody against the foreign body (e.g., dengue IgM antibodies in serum specimen from day 5-6 of illness), or detection of a rise (e.g., two-fold, three-fold, four-fold, five-fold, or more) of a specific IgG antibody titre on a pair of sera (e.g., acute and convalescent specimens). As another example, cross-reactions between a first virus (e.g., a dengue virus) and a related second virus (e.g., a flavivirus) may be performed at appropriate time points to measure a decrease in a foreign body.

For example, the amount of the foreign body (e.g., in the bloodstream of a subject) can decrease by at least about 5% (e.g., at least about 10%, 15%, 20%, 25%, 30%, 40%, 50%, 60%, 70%, 80%, or 90%). The foreign body can be any appropriate foreign body. For example, the foreign body can be a pathogen. A pathogen can be any appropriate pathogen. In some embodiments, the pathogen can be a bacterium (e.g., Staphylococcus aureus), a fungus, a protozoa, or a virus. A bacterium, a fungus, a protozoa, or a virus can be any appropriate bacterium, fungus, protozoa, or virus, for example, any of the bacteria, fungi, protozoa, or viruses described herein. The foreign body can be located in or on the body of the subject in any appropriate location. For example, in some embodiments, the foreign body can be in the bloodstream of the subject. In some embodiments, the foreign body can be on the skin of the subject. In some embodiments, the foreign body can be in a wound (e.g., a surgical wound or a non-surgical wound) of a subject.

Administration of a composition comprising platelets such as lyophilized platelets, or platelet derivatives (e.g., thrombosomes, optionally rehydrated) can be carried out by any appropriate method. For example, administration can be topical administration (e.g., in the form of a spray, a solution, a gel, a cream, or an ointment). In some embodiments, administration can be parenteral administration (e.g., intravenous administration, intramuscular administration, intrathecal administration, subcutaneous administration, or intraperitoneal administration). In some embodiments, administration can be intravenous administration. In some embodiments, administration can be pulmonary administration (e.g., using a particulate inhaler). In some embodiments, administration of a composition comprising platelets such as lyophilized platelets or platelet derivatives (e.g., thrombosomes, optionally rehydrated) can be performed when the subject has previously been administered one or more anticoagulants (e.g., while the anticoagulants are present in an effective dose in the subject).

Incubation of the platelets with an incubating agent may be performed at 37° C., using different incubation periods. The platelets may be suspended in a buffer at a concentration from 10,000 platelets/μL to 10,000,000 platelets/μL, such as 50,000 platelets/μL to 2,000,000 platelets/μL, such as 100,000 platelets/μL to 500,000 platelets/μL, such as 150,000 platelets/μL to 300,000 platelets/μL, such as 200,000 platelets/μL. An exemplary concentration is 200,000 platelets/μl.

Also provided herein are in vitro methods of detecting an interaction between a composition comprising platelets such as lyophilized platelets, or platelet derivatives (e.g., thrombosomes, optionally rehydrated) and an incubating agent comprising one or more salts, a buffer, optionally a cryoprotectant, and optionally an organic solvent and a foreign body. The methods can include combining the composition comprising platelets such as lyophilized platelets, or platelet derivatives (e.g., thrombosomes, optionally rehydrated) and an incubating agent comprising one or more salts, a buffer, optionally a cryoprotectant and the foreign body in an aqueous medium and detecting an interaction between the composition comprising platelets such as lyophilized platelets, or platelet derivatives (e.g., thrombosomes, optionally rehydrated) and an incubating agent comprising one or more salts, a buffer, optionally a cryoprotectant and the foreign body. Detecting can be performed by any appropriate method. In some embodiments, detecting includes using aggregometry. In some embodiments, detecting includes using flow cytometry. In some embodiments, detecting includes using fluorescence microscopy. In some embodiments, the aqueous medium can include a buffer, a salt, optionally a cryoprotectant, and optionally an organic solvent. In some embodiments, the aqueous medium can further include comprises an aggregation agonist. An aggregation agonist can be, in some cases, adenosine diphosphate or collagen. An agonist can be used to assess interactions of activated platelets such as lyophilized platelets, or platelet derivatives (e.g., thrombosomes) with a foreign body of interest. In the absence of an agonist, the potential for a foreign body of interest to activate platelets such as lyophilized platelets, or platelet derivatives (e.g., thrombosomes) can be assessed. In some embodiments, the aqueous medium can further include human plasma fibrinogen. In some embodiments, the foreign body can be a bacterium, a virus, a fungus, or a protozoa (e.g., any of the bacteria, viruses, fungi, or protozoa described herein). In some embodiments, the bacterium can be an antibiotic-resistant bacterium. In some embodiments, the bacterium can be Staphylococcus aureus (e.g., PANSORBIN®). In some embodiments, the foreign body can be supplied as a suspension (e.g., a 5%-20%, 10-15%, 8-12%, or 10% (w/v) suspension) in a buffer (e.g., PBS, optionally with 0.1% NaN₃) at an appropriate pH (e.g., 7.2). The suspension of the foreign body can be about <2.5% v/v of the final sample for the in vitro methods.

These methods allow for the use of pathogens (e.g., nonviable or fixed pathogens) in situations where it may be undesirable to work with living, replicating pathogenic bodies, such as in labs not equipped for the culture and handling of live pathogen. The shelf life of fixed bacteria can be over a year at 4° C., compared to live bacteria which are typically maintained as a live culture or frozen at −80° C. in glycerol for extended storage.

### EXEMPLARY EMBODIMENTS

Embodiment 1 is a method of treating a bacterial infection in a subject, the method comprising administering to the subject in need thereof an effective amount of a composition comprising platelets or platelet derivatives and an incubating agent comprising one or more salts, a buffer, optionally a cryoprotectant, and optionally an organic solvent.

Embodiment 2 is the method of embodiment 1, wherein the bacterial infection is an antibiotic resistant bacterial infection.

Embodiment 3 is the method of embodiment 1 or embodiment 2, wherein the bacterial infection is a Staphylococcus aureus infection.

Embodiment 4 is a method of treating a gram negative bacterial infection in a subject, the method comprising administering to the subject in need thereof an effective amount of a composition comprising platelets or platelet derivatives and an incubating agent comprising one or more salts, a buffer, optionally a cryoprotectant, and optionally an organic solvent.

Embodiment 5 is the method of embodiment 4, wherein the gram negative bacterial infection is caused by E. coli, Pseudomonas aeruginosa, Neisseria gonorrhoeae, Chlamydia trachomatis, Yersinia pestis, or two or more thereof.

Embodiment 6 is a method of treating a gram positive bacterial infection in a subject, the method comprising administering to the subject in need thereof an effective amount of a composition comprising platelets or platelet derivatives and an incubating agent comprising one or more salts, a buffer, optionally a cryoprotectant, and optionally an organic solvent.

Embodiment 7 is the method of embodiment 6, wherein the gram positive bacterial infection is caused by Bacillus anthracis, Corynebacterium diphtherias, Enterococcus faecalis, Enterococcus faecium, Erysipelothrix rhusiopathiae, Listeria monocytogenes, Streptococcus pneumonia, Staphylococcus aureus, Streptococcus pyogenes, Streptococcus agalactiae, Viridans streptococci, or two or more thereof.

Embodiment 8 is a method of treating a fungal infection in a subject, the method comprising administering to the subject in need thereof an effective amount of a composition comprising platelets or platelet derivatives such as freeze-dried platelets and an incubating agent comprising one or more salts, a buffer, optionally a cryoprotectant, and optionally an organic solvent.

Embodiment 9 is the method of embodiment 8, wherein the fungal infection is caused by an Aspergillus species, a Blastomyces species, a Candida species, a Coccidioides species, a Cryptococcus species, a Histoplasma species, a Pneumocystis species, or two or more thereof.

Embodiment 10 is a method of treating protozoan infection in a subject, the method comprising administering to the subject in need thereof an effective amount of a composition comprising platelets or platelet derivatives an incubating agent comprising one or more salts, a buffer, optionally a cryoprotectant, and optionally an organic solvent.

Embodiment 11 is the method of embodiment 10, wherein the protozoan infection is caused by an Entamoeba species, a Plasmodium species, a Giardia species, a Trypanosoma species, a Leishmania species, a Toxoplasma species, or two or more thereof.

Embodiment 12 is a method of treating a viral infection in a subject, the method comprising administering to the subject in need thereof an effective amount of a composition comprising platelets or platelet derivatives and an incubating agent comprising one or more salts, a buffer, optionally a cryoprotectant, and optionally an organic solvent.

Embodiment 13 is the method of embodiment 12, wherein the viral infection is caused by a member of one or more of the following families: Adenoviridae, Herpesviridae, Papillomaviridae, Polyomaviridae, Poxviridae, Hepadnaviridae, Parvoviridae, Astroviridae, Caliciviridae, Picornaviridae, Coronaviridae, Flaviviridae, Togaviridae, Hepeviridae, Retroviridae, Orthomyxoviridae, Arenaviridae, Bunyaviridae, Filoviridae, Paramyxoviridae, Rhabdoviridae, or Reoviridae.

Embodiment 14 is the method of embodiment 12, wherein the viral infection is caused by a human immunodeficiency virus (HIV).

Embodiment 15 is the method of embodiment 13, wherein the viral infection is caused by a member of the Coronaviridae family.

Embodiment 16 is the method of embodiment 15, wherein the viral infection is caused by a Betacoronavirus.

Embodiment 17 is the method of embodiment 16, wherein the Betacoronavirus is selected from the group consisting of SaRS-CoV, MERS-CoV, SaRS-CoV-2, or a combination thereof.

Embodiment 18 is a method of treating a hemorrhagic viral infection in a subject, the method comprising administering to the subject in need thereof an effective amount of a composition comprising platelets or platelet derivatives and an incubating agent comprising one or more salts, a buffer, optionally a cryoprotectant, and optionally an organic solvent.

Embodiment 19 is the method of embodiment 18, wherein the hemorrhagic viral infection is caused by is Ebola virus, Marburg Virus, Lassa virus, or Dengue virus.

Embodiment 20 is a method of treating a non-hemorrhagic viral infection in a subject, the method comprising administering to the subject in need thereof an effective amount of a composition comprising platelets or platelet derivatives and an incubating agent comprising one or more salts, a buffer, optionally a cryoprotectant, and optionally an organic solvent.

Embodiment 21 is the method of embodiment 20, wherein the non-hemorrhagic viral infection is caused by a member of one or more of the following families: Adenoviridae, Herpesviridae, Papillomaviridae, Polyomaviridae, Poxviridae, Hepadnaviridae, Parvoviridae, Astroviridae, Caliciviridae, Picornaviridae, Coronaviridae, Flaviviridae, Togaviridae, Hepeviridae, Retroviridae, Orthomyxoviridae, Arenaviridae, Bunyaviridae, Paramyxoviridae, Rhabdoviridae, or Reoviridae.

Embodiment 22 is a method of binding a foreign body in a subject, the method comprising administering to the subject in need thereof an effective amount of a composition comprising platelets or platelet derivatives and an incubating agent comprising one or more salts, a buffer, optionally a cryoprotectant, and optionally an organic solvent, wherein the foreign body binds to the platelets or platelet derivatives, such as freeze-dried platelets.

Embodiment 23 is the method of embodiment 22, wherein the foreign body is a pathogen.

Embodiment 24 is the method of embodiment 22 or embodiment 23, wherein the foreign body is a bacterium.

Embodiment 25 is the method of embodiment 24, wherein the foreign body is Staphylococcus aureus.

Embodiment 26 is the method of embodiment 22 or embodiment 23, wherein the foreign body is a fungus.

Embodiment 27 is the method of embodiment 22 or embodiment 23, wherein the foreign body is a protozoa.

Embodiment 28 is the method of embodiment 22 or embodiment 23, wherein the foreign body is a virus.

Embodiment 29 is the method of embodiment 28, wherein the virus is Ebola virus, Marburg Virus, Lassa virus, or Dengue virus.

Embodiment 30 is the method of embodiment 28, wherein the virus is a human immunodeficiency virus.

Embodiment 31 is the method of embodiment 28, wherein the virus is a member of Coronaviridae.

Embodiment 32 is the method of embodiment 28, wherein the virus is a member of Betacoronavirus.

Embodiment 33 is the method of embodiment 28, wherein the virus is selected from the group consisting of SaRS-CoV, MERS-CoV, SaRS-CoV-2, or a combination thereof.

Embodiment 34 is the method of any one of embodiments 22-33, wherein the foreign body is in the bloodstream of the subject.

Embodiment 35 is the method of any one of embodiments 22-33, wherein the foreign body is on the skin of the subject.

Embodiment 36 is a method of binding a foreign body in the bloodstream of a subject, the method comprising administering to the subject in need thereof an effective amount of a composition comprising platelets or platelet derivatives and an incubating agent comprising one or more salts, a buffer, optionally a cryoprotectant, and optionally an organic solvent, wherein the amount of the foreign body in the bloodstream of the subject decreases by at least 5%.

Embodiment 37 is the method of embodiment 36, wherein the foreign body is a pathogen.

Embodiment 38 is the method of embodiment 36 or embodiment 37, wherein the foreign body is a bacterium.

Embodiment 39 is the method of embodiment 38, wherein the foreign body is Staphylococcus aureus.

Embodiment 40 is the method of embodiment 36 or embodiment 37, wherein the foreign body is a fungus.

Embodiment 41 is the method of embodiment 36 or embodiment 37, wherein the foreign body is a protozoa.

Embodiment 42 is the method of embodiment 36 or embodiment 37, wherein the foreign body is a virus.

Embodiment 43 is the method of embodiment 42, wherein the virus is Ebola virus, Marburg Virus, Lassa virus, or Dengue virus.

Embodiment 44 is the method of embodiment 36, wherein the virus is a human immunodeficiency virus.

Embodiment 45 is the method of embodiment 42, wherein the virus is a member of Coronaviridae.

Embodiment 46 is the method of embodiment 42, wherein the virus is a member of Betacoronavirus.

Embodiment 47 is the method of embodiment 42, wherein the virus is selected from the group consisting of SaRS-CoV, MERS-CoV, SaRS-CoV-2, or a combination thereof.

Embodiment 48 is the method of any one of embodiments 36-47, wherein the amount of the foreign body in the bloodstream of the subject decreases by at least 10%.

Embodiment 49 is the method of any one of embodiments 36-47, wherein the amount of the foreign body in the bloodstream of the subject decreases by at least 20%.

Embodiment 50 is the method of any one of embodiments 1 to 49, wherein the platelets or platelet derivatives have been freeze-dried.

Embodiment 51 is the method of embodiment 50, wherein the platelets or platelet derivatives have been rehydrated.

Embodiment 52 is the method of any one of embodiments 1 to 51, wherein the incubating agent comprises one or more salts selected from phosphate salts, sodium salts, potassium salts, calcium salts, magnesium salts, and a combination of two or more thereof.

Embodiment 53 is the method of any one of embodiments 1 to 52, wherein the concentration of the one or more salts is from about 0.5 mM to about 100 mM.

Embodiment 54 is the method of any one of embodiments 1 to 53, wherein the buffer comprises HEPES, sodium bicarbonate (NaHCO₃), or a combination thereof.

Embodiment 55 is the method of any one of embodiments 1 to 54, wherein the concentration of the buffer is from about 5 to about 50 mM.

Embodiment 56 is the method of any one of embodiments 1 to 55, wherein the composition comprises one or more saccharides.

Embodiment 57 is the method of embodiment 56, wherein the one or more saccharides comprise trehalose.

Embodiment 58 is the method of embodiment 56 or 57, wherein the one or more saccharides comprise polysucrose.

Embodiment 59 is the method of any one of embodiments 1-58, wherein administering comprises administering topically.

Embodiment 60 is the method of any one of embodiments 1-58, wherein administering comprises administering parenterally.

Embodiment 61 is the method of any one of embodiments 1-58, wherein administering comprises administering intravenously.

Embodiment 62 is the method of any one of embodiments 1-58, wherein administering comprises administering intramuscularly.

Embodiment 63 is the method of any one of embodiments 1-58, wherein administering comprises administering intrathecally.

Embodiment 64 is the method of any one of embodiments 1-58, wherein administering comprises administering subcutaneously.

Embodiment 65 is the method of any one of embodiments 1-58, wherein administering comprises administering intraperitoneally.

Embodiment 66 is the method of any one of embodiments 1-58, wherein administering comprises administering to the pulmonary system.

Embodiment 67 is a method of treating an infection in a subject, the method comprising administering to the subject in need there of an effective amount of a composition comprising platelets or platelet derivatives prepared by incubating platelets with an incubating agent comprising one or more salts, a buffer, optionally a cryoprotectant, and optionally an organic solvent.

Embodiment 68 is the method of embodiment 67, wherein the infection is a bacterial infection.

Embodiment 69 is the method of embodiment 68, wherein the bacterial infection is an antibiotic-resistant bacterial infection.

Embodiment 70 is the method of embodiment 68 or 69, wherein the bacterial infection is a Staphylococcus aureus infection.

Embodiment 71 is the method of embodiment 67, wherein the infection is a fungal infection.

Embodiment 72 is the method of embodiment 67, wherein the infection is a protozoan infection.

Embodiment 73 is the method of embodiment 67, wherein the infection is a viral infection.

Embodiment 74 is the method of embodiment 73, wherein the virus is Ebola virus, Marburg Virus, Lassa virus, or Dengue virus.

Embodiment 75 is the method of embodiment 73, wherein the virus is a human immunodeficiency virus.

Embodiment 76 is the method of embodiment 73, wherein the virus is a member of Coronaviridae.

Embodiment 77 is the method of embodiment 73, wherein the virus is a member of Betacoronavirus.

Embodiment 78 is the method of embodiment 73, wherein the virus is selected from the group consisting of SaRS-CoV, MERS-CoV, SaRS-CoV-2, or a combination thereof.

Embodiment 79 is the method of any one of embodiments 67-78, wherein the infection is in the bloodstream of the subject.

Embodiment 80 is the method of any one of embodiments 67-78, wherein the infection is on the skin of the subject.

Embodiment 81 is the method of any one of embodiments 67-78, wherein the salt comprises a phosphate salt, a sodium salt, a potassium salt, a calcium salt, a magnesium salt, or a combination thereof.

Embodiment 82 is the method of any one of embodiments 67-81, wherein the salt comprises sodium chloride, potassium chloride, or a combination thereof.

Embodiment 83 is the method of any one of embodiments 67-82, wherein the salt is present in an amount of about 0.5 mM to about 100 mM.

Embodiment 84 is the method of any one of embodiments 67-82, wherein the salt is present in an amount of about 2 mM to about 6 mM.

Embodiment 85 is the method of any one of embodiments 67-82, wherein the salt is present in an amount of about 60 mM to about 90 mM.

Embodiment 86 is the method of any one of embodiments 67-85, wherein the buffer comprises N-2-hydroxyethylpiperazine-N′-2- ethanesulfonic acid, sodium bicarbonate, or a combination thereof.

Embodiment 87 is the method of any one of embodiments 67-86, wherein the buffer is present in an amount of about 5 to about 50 mM.

Embodiment 88 is the method of any one of embodiments 67-87, wherein the buffer is present in an amount of about 10 to about 25 mM.

Embodiment 89 is the method of any one of embodiments 67-88, wherein the cryoprotectant comprises a saccharide.

Embodiment 90 is the method of embodiment 89, wherein the saccharide comprises a monosaccharide, a disaccharide, or a combination thereof.

Embodiment 91 is the method of embodiment 89 or embodiment 90, wherein the saccharide comprises sucrose, maltose, trehalose, glucose, mannose, dextrose, xylose, or a combination thereof.

Embodiment 92 is the method of any one of embodiments 67-91, wherein the cryoprotectant comprises polysucrose.

Embodiment 93 is the method of any one of embodiments 67-92, wherein the cryoprotectant is present in an amount of about 10 mM to about 1000 mM.

Embodiment 94 is the method of any one of embodiments 67-93, wherein the cryoprotectant is present in an amount of about 50 mM to about 200 mM.

Embodiment 95 is the method of any one of embodiments 67-94, wherein the organic solvent comprises ethanol, DMSO, or a combination thereof

Embodiment 96 is the method of any one of embodiments 67-95, wherein the organic solvent is present in an amount of about 0.1% (v/v) to about 5.0% (v/v).

Embodiment 97 is the method of any one of embodiments 67-95, wherein the organic solvent is present in an amount of about 0.3% (v/v) to about 3.0% (v/v).

Embodiment 98 is the method of any one of embodiments 67-97, wherein the incubating agent further comprises a carrier protein.

Embodiment 99 is the method of embodiment 98, wherein the carrier protein comprises human serum albumin, bovine serum albumin, or a combination thereof.

Embodiment 100 is the method of embodiment 98 or embodiment 99, wherein the carrier protein is present in an amount of about 0.05% to about 1.0% (w/v).

Embodiment 101 is the method of any one of embodiments 67-100, wherein the incubating agent comprises Ca²⁺, Mg²⁺, or a combination thereof.

Embodiment 102 is the method of embodiment 101, wherein the Ca²⁺, the Mg²⁺, or a combination thereof is present in an amount of about 0.5 mM to about 2 mM.

Embodiment 103 is the method of any one of embodiments 67-102, wherein the process for preparing the composition comprising platelets and an incubating agent comprises drying a composition comprising platelets and an incubating agent.

Embodiment 104 is the method of embodiment 103, wherein drying comprises freeze-drying.

Embodiment 105 is the method of embodiment 103 or embodiment 104, wherein the process for preparing the composition comprising platelets and an incubating agent further comprises heating the composition comprising platelets and an incubating agent.

Embodiment 106 is the method of embodiment 105, wherein heating the composition comprising platelets and an incubating agent comprises heating at about 75° C. to about 85° C. for about 24 hours.

Embodiment 107 is the method of any one of embodiments 103-105, wherein the process for preparing the composition comprising platelets and an incubating agent comprises rehydrating a composition comprising platelets and an incubating agent.

Embodiment 108 is the method of embodiment 107, wherein the rehydrating comprises rehydrating with water.

Embodiment 109 is the method of embodiment 107, wherein the rehydrating comprises rehydrating with a buffer.

Embodiment 110 is the method of any one of embodiments 1-109, wherein the platelet derivatives comprise thrombosomes.

Embodiment 111 is the method of any one of embodiments 1-110, wherein the platelets comprise lyophilized platelets.

Embodiment 112 is an in vitro method of detecting an interaction between comprising platelets or platelet derivatives and an incubating agent comprising one or more salts, a buffer, optionally a cryoprotectant and a foreign body, the method comprising:
combining:
   a composition comprising platelets or platelet derivatives and an incubating agent comprising one or more salts, a buffer, optionally a cryoprotectant, and optionally an organic solvent,
   a foreign body, and
   an aqueous medium; and detecting an interaction between the composition and the foreign body.

Embodiment 113 is the in vitro method of embodiment 112, wherein detecting comprises using aggregometry.

Embodiment 114 is the in vitro method of embodiment 112, wherein detecting comprises using flow cytometry.

Embodiment 115 is the in vitro method of embodiment 112, wherein detecting comprises using fluorescence microscopy.

Embodiment 116 is the in vitro method of any one of embodiments 112-115, wherein the platelets or platelet derivatives were previously freeze-dried.

Embodiment 117 is the in vitro method of embodiment 116, wherein the platelets or platelet derivatives were rehydrated after being freeze-dried.

Embodiment 118 is the in vitro method of any one of embodiments 111-117, wherein the aqueous medium comprises a buffer, a salt, optionally a cryoprotectant, and optionally an organic solvent.

Embodiment 119 is the in vitro method of embodiment 118, wherein the aqueous medium further comprises an aggregation agonist.

Embodiment 120 is the in vitro method of embodiment 119, wherein the aggregation agonist comprises adenosine diphosphate or collagen.

Embodiment 121 is the in vitro method of any one of embodiments 112-120, wherein the aqueous medium further comprises human fibrinogen.

Embodiment 122 is the in vitro method of any one of embodiments 112-121, wherein the foreign body is a bacterium, a virus, a fungus, or a protozoa.

Embodiment 123 is the in vitro method of embodiment 122, wherein the bacterium is an antibiotic-resistant bacterium.

Embodiment 124 is the in vitro method of embodiment 122 or embodiment 123, wherein the bacterium Staphylococcus aureus.

Embodiment 125 is the in vitro method of embodiment 122, wherein the virus is Ebola virus, Marburg Virus, Lassa virus, or Dengue virus.

Embodiment 126 is the in vitro method of embodiment 122, wherein the virus is a human immunodeficiency virus.

Embodiment 127 is the method of embodiment 122, wherein the virus is a member of Coronaviridae.

Embodiment 128 is the method of embodiment 122, wherein the virus is a member of Betacoronavirus.

Embodiment 129 is the method of embodiment 122, wherein the virus is selected from the group consisting of SaRS-CoV, MERS-CoV, SaRS-CoV-2, or a combination thereof.

Embodiment 130 is the in vitro method of any one of embodiments 112-129, wherein the platelet derivatives comprise thrombosomes.

Embodiment 131 is the in vitro method of any one of embodiments 112-130, wherein the platelets comprise lyophilized platelets.

### EXAMPLES

Example 1

Heat-killed, formalin fixed S. aureus was used to model the interactions of S. aureus with platelets and platelet derivatives, such as freeze-dried platelet derivatives, as disclosed herein.

In particular, a heat-killed, formalin fixed S. aureus of strain Cowan 1 (e.g., PANSORBIN® cells; see, e.g., MilliporeSigma catalog number 507858) were used to model such physical interactions in vitro. PANSORBIN® can be safely handled in the general laboratory environment and does not require cell culture capabilities or specialized storage conditions for extended study. The described bacteria are commercially sold under the name PANSORBIN® for use in antibody purification, mitogenic stimulation of B cells, and immunoprecipitation applications.

Thrombosomes as disclosed herein (e.g., including trehalose and polysucrose in the incubating agent) were suspended in an aqueous medium. The aqueous medium contained approximately 1 mg/mL human plasma fibrinogen, approximately 1 mM Ca²⁺, and approximately 1 mM Mg²⁺. Human plasma and/or human whole blood were optional components. An aqueous buffer was supplemented with appropriate concentrations of human plasma fibrinogen, Ca²⁺, and Mg²⁺. PANSORBIN® was supplied as a 10% w/v suspension of fixed cells in PBS with 0.1% NaN₃ at pH 7.2. This PANSORBIN® suspension was added to the platelet medium such that the PANSORBIN® suspension constituted ≤2.5% v/v of the final sample.

The in vitro study was performed using washed platelets at a concentration of 250*10³/uL or two compositions as described below. A first composition had a concentration of platelets of 300*10³/uL and a composition containing the components of Table 6. The composition of Table 6, not including the platelets or thrombosomes, is referred to herein as “HMTA composition”, where HMTA stands for “HEPES modified Tyrode's albumin”:

TABLE 6

| | Concentration |
|---|---|
| Component | (mM, except where otherwise indicated) |
| HEPES | 9.5 |
| NaCl | 145.0 |
| KCl | 4.8 |
| NaHCO₃ | 12.0 |
| Dextrose | 5.0 |
| Bovine Serum Albumin | 0.35% w/v |

A second composition had a concentration of platelets of 300*10³/uL and a composition containing the components of Table 7. The composition of Table 7, not including the platelets or thrombosomes, is referred to herein as “supplemented HMTA”:

TABLE 7

| | Concentration | |
|---|---|---|
| Component | (mM, except where otherwise indicated) | |
| HEPES | 9.5 | |
| NaCl | 145.0 | |
| KCl | 4.8 | |
| NaHCO₃ | 12.0 | |
| Dextrose | 5.0 | |
| Bovine Serum Albumin | 0.35% | w/v |
| Human Plasma Fibrinogen | 1.0 | mg/mL |

| | |
|---|---|
| CaCl₂ | 1.0 |
| MgCl₂ | 1.0 |

Results for the given testing conditions are provided below. PANSORBIN® interactions were evaluated with (1) platelets suspended in supplemented HTMA, (2) a composition containing thrombosomes at a final concentration of 300*10³/uL, and the components of Table 6 (“composition (2)”), and (3) a composition containing thrombosomes at a final concentration of 300*10³/uL and the components of Table 7 (“composition (3)”). For both (2) and (3), the compositions were first freeze-dried, and then rehydrated to obtain the concentrations shown in the appropriate Table.

As used hereinbelow, “fresh platelets” are defined as platelets isolated by centrifugal fractionation from whole blood drawn into ACD and used within 3 hours of collection. “Stored platelets” are defined as platelets collected by the apheresis method into ACD and stored at room temperature in a gas-permeable bag for up to 48 hours post-collection.

The PANSORBIN® interactions with platelets, compositions (2) and (3) were evaluated using Light Transmission Aggregometry on a Helena AggRAM Aggregometer, flow cytometry on a BD Accuri C6 Plus Flow Cytometer, and by fluorescence microscopy on an Olympus BX43 Microscope.

Platelet aggregation agonists adenosine diphosphate (ADP; 20 final concentration) and collagen (10 μg/mL, final concentration) were used in some cases. Phosphate-buffered saline was used as a control.

A strong aggregation response measured by light transmission aggregometry in the presence of platelets (e.g., freeze-dried platelets) and PANSORBIN®, particularly in the absence of a platelet aggregation agonist, indicates incorporation of the PANSORBIN® cells into an aggregation of platelets, composition (2) or (3).

Co-localization of PANSORBIN® and platelets, composition (2) or (3) can be measured using fluorescent staining with detection by flow cytometry and fluorescence microscopy. The surface stain BODIPY (e.g., BODIPY FL Maleimide) is used to dye PANSORBIN®, while biotin and streptavidin-Dylight633 are used to stain the platelets or the thrombosomes in compositions (2) or (3). Colocalization of the two fluorescence signals is indicative of physical binding. PANSORBIN® is smaller than platelets or thrombosomes (1 μm diameter vs 2-3 μm diameter, respectively) and this difference is readily apparent by microscopy.

TABLE 8

| | | | | Max % | Apparent |
|---|---|---|---|---|---|
| Channel | Component 1 | Component 2 | Agonist | Agg | Lag Time |
| 1 | Platelets | PBS | PBS | 19.2 | |
| 2 | Platelets | PBS | ADP | 84.9 | |
| 3 | Platelets | PBS | Collagen | 94.6 | |
| 4 | Platelets | PANSORBIN ® | PBS | 84.2 | 280 s |
| 5 | Platelets | PANSORBIN ® | ADP | 80.6 | |
| 6 | Platelets | PANSORBIN ® | Collagen | 86.0 | |
| | Supplemented | | | | |
| 7 | HMTA | PANSORBIN ® | PBS | 6.5 | |
| | Supplemented | | | | |
| 8 | HMTA | PANSORBIN ® | Collagen | 38.6 | |

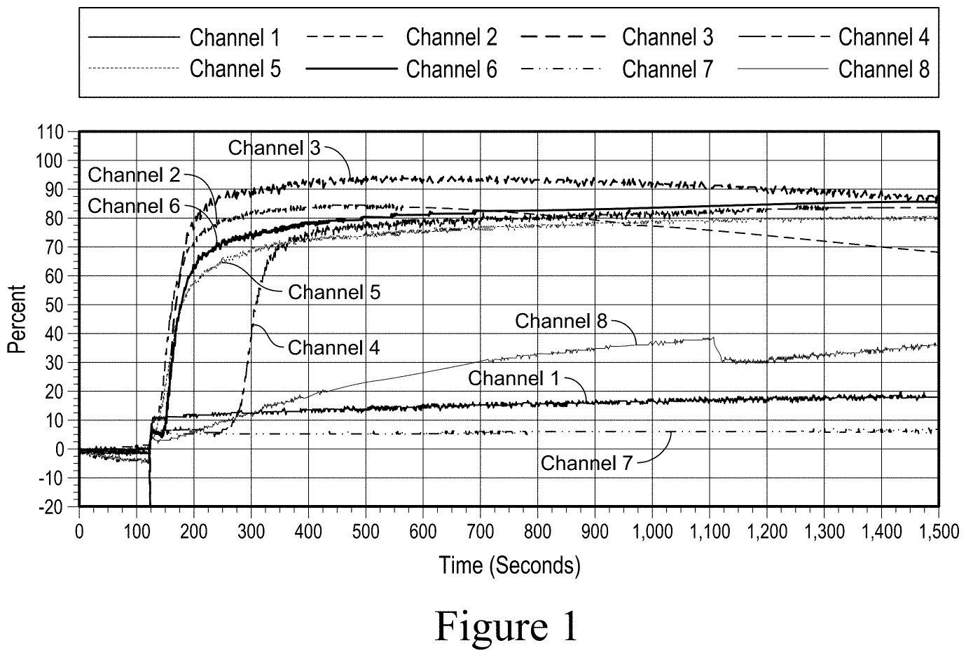

Table 8 shows the experimental layout of the investigation of PANSORBIN® interactions with fresh platelets (or control supplemented HTMA), including the channel layout used in the aggregometer. Platelets and PANSORBIN® were co-incubated in the aggregometry cuvette. Results are shown in FIG. 1. PBS, ADP, or collagen agonist is added at 120 seconds. PANSORBIN® readily co-aggregated with activated fresh platelets, as evidenced by nearly immediate complete light transmittance upon addition of an agonist (ADP 20 μM; collagen 10 μg/mL). PANSORBIN® activated fresh platelets with a lag time of approximately 300 seconds, consistent with many S. aureus strains in the literature record (see, e.g., Miajlovic, H et al. Immunity. 2007; 75(7); 3335-3343. doi:10.1128/IAI.01993-06.)

TABLE 9

| | | | | Max % |
|---|---|---|---|---|
| Channel | Component 1 | Component 2 | Agonist | Agg |
| 1 | Platelets | PBS | ADP | 24.4 |
| 2 | Platelets | PBS | Collagen | 88.3 |
| 3 | Platelets | PANSORBIN ® | ADP | 81.0 |
| 4 | Platelets | PANSORBIN ® | ADP | 78.8 |
| 5 | Platelets | PANSORBIN ® | Collagen | 53.5 |
| 6 | Platelets | PANSORBIN ® | Collagen | 43.3 |
| | Supplemented | | | |
| 7 | HMTA | PANSORBIN ® | Collagen | 9.7 |
| 8 | Platelets | PANSORBIN ® | PBS | 20.6 |

Table 9 shows the experimental layout of the investigation of PANSORBIN® interactions with stored platelets (or control supplemented HTMA), including the channel layout.

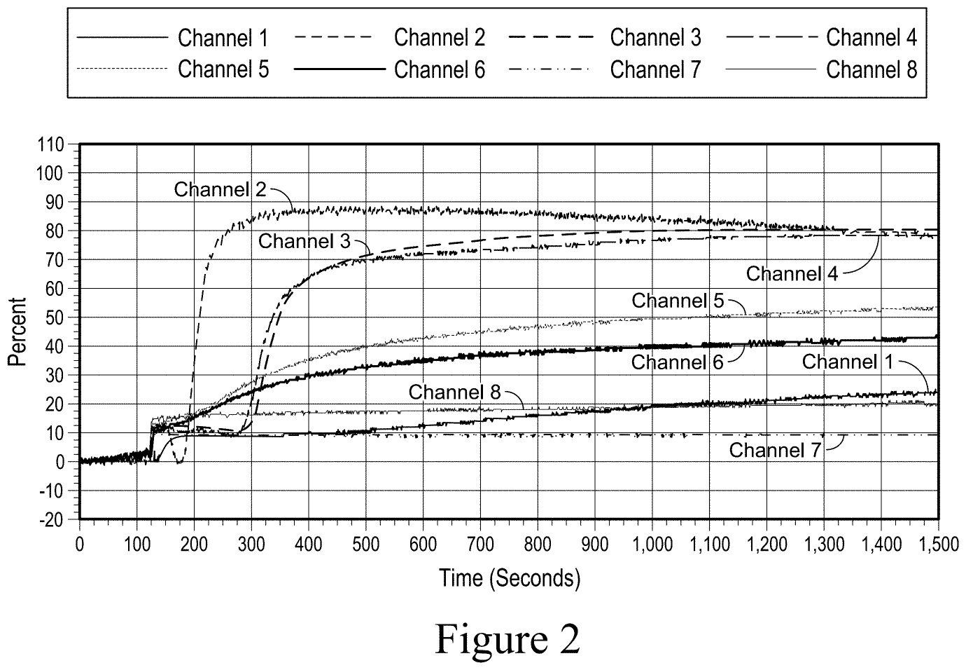

Results are shown in FIG. 2. PANSORBIN® co-aggregated with stored platelets activated with ADP (20 μM) and to a lesser extent collagen (10 μg/mL). PANSORBIN® were not able to activate and aggregate with stored platelets in the absence of an additional agonist. There was an extended lag time between introduction of agonist and induction of aggregation and PANSORBIN® binding, but there was nearly complete incorporation of PANSORBIN® in the platelets with ADP stimulation.

TABLE 10

| Channel | Composition | PANSORBIN ® | Max % Agg |
|---|---|---|---|
| 1 | (2) | PBS | 8.0 |
| 2 | (3) | PBS | 39.6 |
| 3 | PBS + HTMA control | PANSORBIN ® | 2.9 |
| | PBS + supplemented | | |
| 4 | HTMA control | PANSORBIN ® | 24.4 |
| 5 | (2) | PANSORBIN ® | 8.5 |
| 6 | (3) | PANSORBIN ® | 31.5 |
| 7 | (3) | PANSORBIN ® | 31.2 |
| 8 | (3) | PANSORBIN ® | 31.5 |

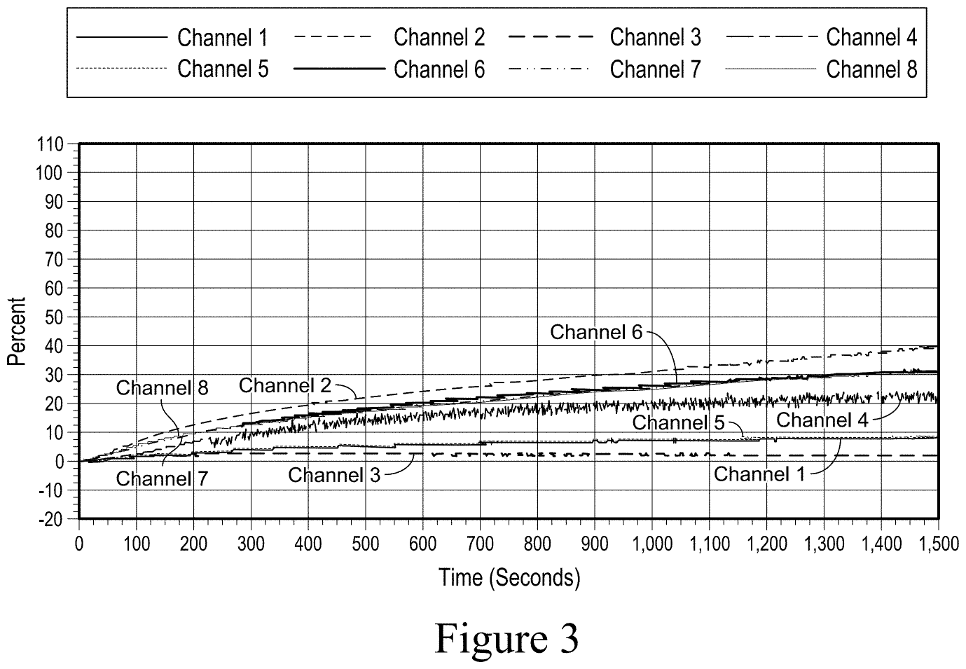

Table 10 shows the experimental layout of the investigation of Compositions (2) (comprising thrombosomes and HMTA) and (3) (comprising thrombosomes and supplemented HMTA) with PANSORBIN®. Results are shown in FIG. 3. Agglutination was exhibited as indicated by a steadily and slowly decreasing optical density as particles associate in solution. Both the composition (3) and PANSORBIN® alone agglutinate in the presence of fibrinogen (channels 2 and 4, respectively), suggesting that co-agglutination is mediated by fibrinogen bridging. Fibrinogen bridging interactions with platelets have been described elsewhere (see, e.g., Kerrigan, The non-thrombotic role of platelets in health and disease; Chapter 4: Platelet interactions with bacteria. 2015. doi: 10.5772/60531; Kerrigan, et al., Molecular basis for Staphylococcus aureus mediated platelet aggregate formation under arterial shear in vitro. Arteriosclerosis Thrombosis and Vascular Biology. 2008; 28(2); 335-340. DOI: 10.1161/ATVBAHA.107.152058; Cox, et al. Platelets and the innate immune system: mechanisms of bacterial-induced platelet activation. Journal of Thrombosis and Haemostasis. 2011; 9; 1097-1107. DOI: 10.1111/j.1538-7836.2011.04264.x; O'Brien, et al., Multiple mechanisms for the activation of human platelet aggregation by Staphylococcus aureus: roles for the clumping factors ClfA and ClfB, the serine-aspartate repeat protein SdrE and protein A. Molecular Microbiology. 2002; 44(4); 1033-1044. doi: 10.1046/j.1365-2958.2002.02935.x; and Miajlovic, et al., Both complement- and fibrinogen-dependent mechanisms contribute to platelet aggregation mediated by Staphylococcus aureus clumping factor B. Infection and Immunity. 2007; 75(7); 3335-3343. doi:10.1128/IAI..01993-06.). The magnitude of co-agglutination is less than that observed in fresh platelets or activated stored platelets, but the co-agglutinated compositions (2) or (3) (as applicable) and PANSORBIN® are visible by eye as small flakes in the suspension. This experiment was performed 1 hour after rehydration of the composition comprising thrombosomes and the applicable buffer.

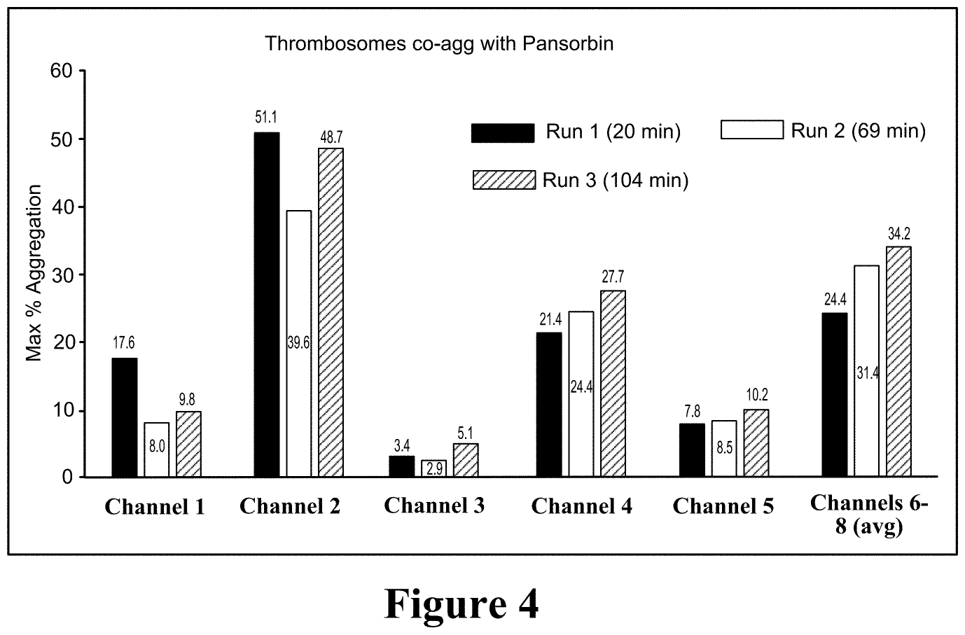

FIG. 4 is a bar graph illustrating a time course with maximum percent aggregation of compositions as described in Table 10 over a 30 minute incubation in the aggregometer. The time indicated in the legend is time elapsed post-rehydration of the dried compositions when the initial suspension optical density (OD) is recorded by the AggRAM. PANSORBIN® and the composition (2) contributed approximately equally to the suspension optical density at the concentrations used. Both the compositions (3) and PANSORBIN® alone agglutinate in the presence of fibrinogen (channels 2 and 4), suggesting that co-agglutination is mediated by fibrinogen bridging.

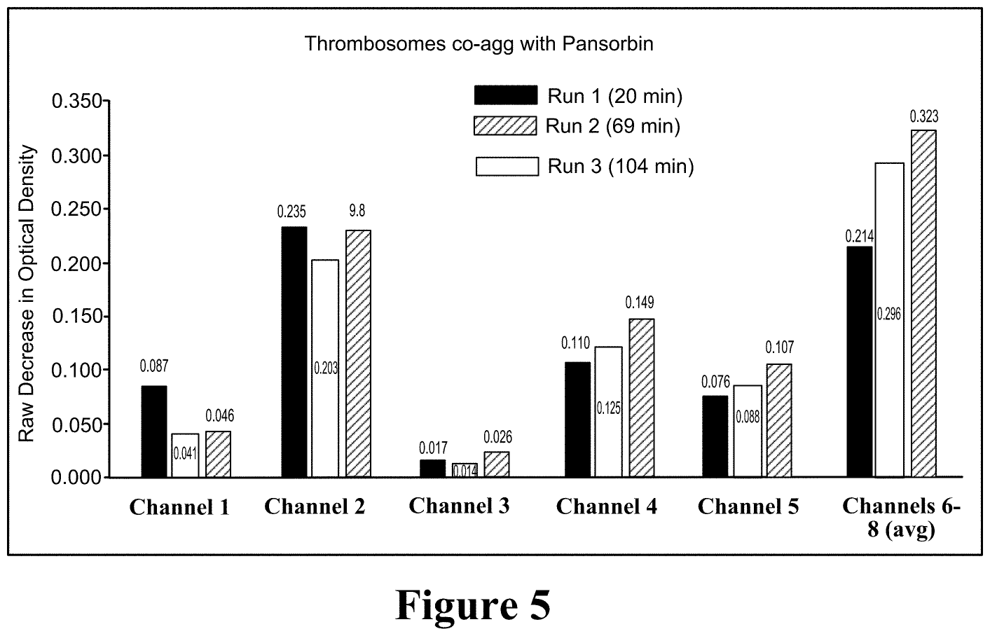

FIG. 5 is a bar graph illustrating a time course with raw change in optical density of compositions as described in Table 10. The time indicated in the legend is time elapsed post- rehydration of the dried compositions when the initial suspension OD is recorded by the AggRAM. PANSORBIN® and the composition (2) contributed approximately equally to the suspension optical density at the concentrations used. The combination of the composition (3) and PANSORBIN® in supplemented HMTA had an almost additive effect on the change in optical density of the suspension over a 30 minute incubation in the aggregometer. However, aggregometry data alone may not be sufficient to distinguish independent agglutination from co-agglutination of thrombosomes and PANSORBIN®.

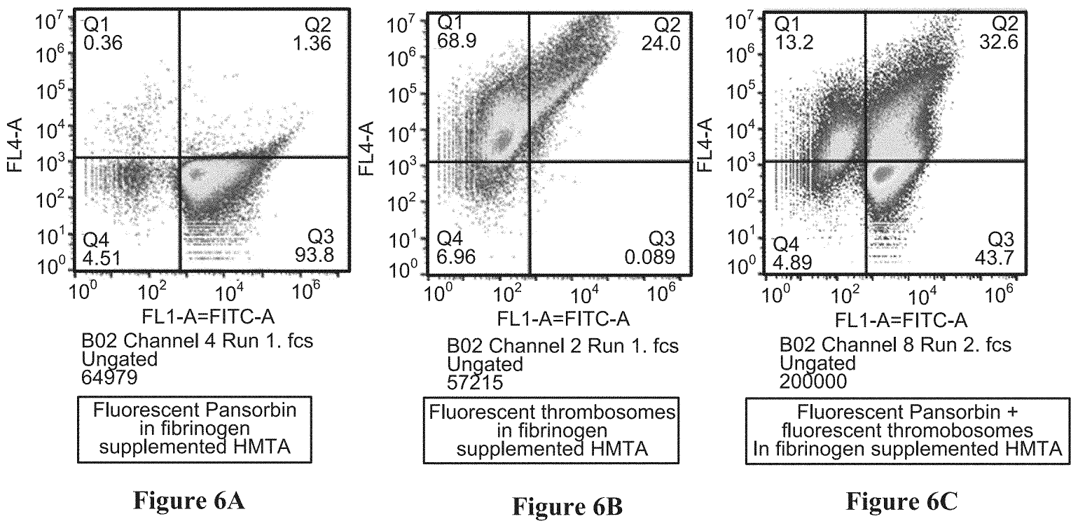

Therefore, flow cytometry experiments were carried out. FIG. 6 shows flow cytometric results of co-agglutination using BODIPY-stained PANSORBIN® (FIG. 6A; FL-1 (488 nm excitation, 533 nm detection with a 30 nm width); x-axis) and Streptavidin-Dylight633-stained thrombosomes in composition (3) (FIG. 6B; FL-4 (640 nm excitation, 675 nm detection with a 25 nm width; y-axis). Gates were placed such that there is maximum separation between the two independent populations. FIG. 6A shows that fluorescent PANSORBIN® in supplemented HMTA were gated into quadrant 3: FL-1 positive and FL-4 negative. FIG. 6B shows that biotin-labeled and Streptavidin-Dylight633-stained thrombosomes in composition (3) were gated into Q1: FL-1 negative and FL-4 positive. Because the thrombosomes in composition (3) are autofluorescent in FL-1, larger composition (3) aggregates appeared in Q2 (double positive) due to magnification of this autofluorescence signal. FIG. 6C shows that after labeled PANSORBIN® and composition (3) were coincubated 30 minutes in the aggregometer, coincident events were detected in Q2. A mathematical correction may be applied to remove false positive events due to composition (3) autofluorescence and the remaining events are interpreted as PANSORBIN® cells associated with thrombosomes in composition (3). In a total sample collection of 200,000 events, approximately 25% of them are such (n=12).

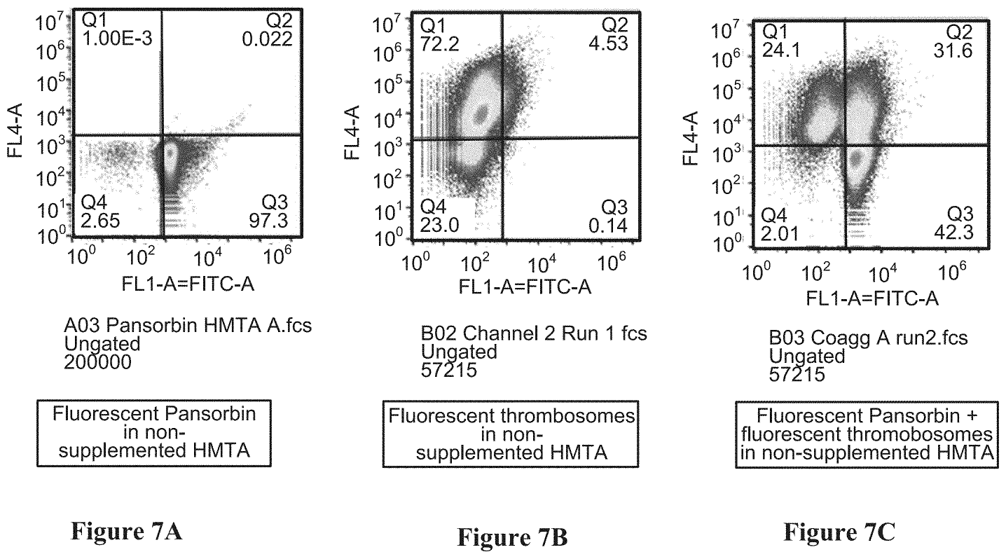

FIG. 7 shows cytometric results of co-agglutination using BODIPY-stained PANSORBIN® (FIG. 7A; FL-1; x-axis) and Streptavidin-Dylight633-stained thrombosomes in composition (2) thrombosomes (FIG. 7B; FL-4; y-axis). Gates were placed such that there is maximum separation between the two independent populations. FIG. 6A shows that fluorescent PANSORBIN® in HMTA (non-supplemented) were gated into Q3: FL-1 positive and FL-4 negative. FIG. 6B shows that biotin-labeled and Streptavidin-Dylight633-stained thrombosomes in composition (2) were gated into Q1: FL-1 negative and FL-4 positive. Fewer false positives appear in Q2 with composition (2) than with composition (3) due to decreased fibrinogen bridging. FIG. 6C shows that after labeled PANSORBIN® and composition (2) were coincubated 30 minutes in the aggregometer, coincident events were detected in Q2. After mathematical correction the remaining events are interpreted as PANSORBIN® cells associated with thrombosomes in composition (2) (approximately 30%, n=2). Supplemental fibrinogen and cations are not necessary for PANSORBIN® binding to composition (2), suggesting PANSORBIN® may bind to residual fibrinogen that has been immobilized onto the surface of thrombosomes in composition (2) during the lyophilization and rehydration processes.

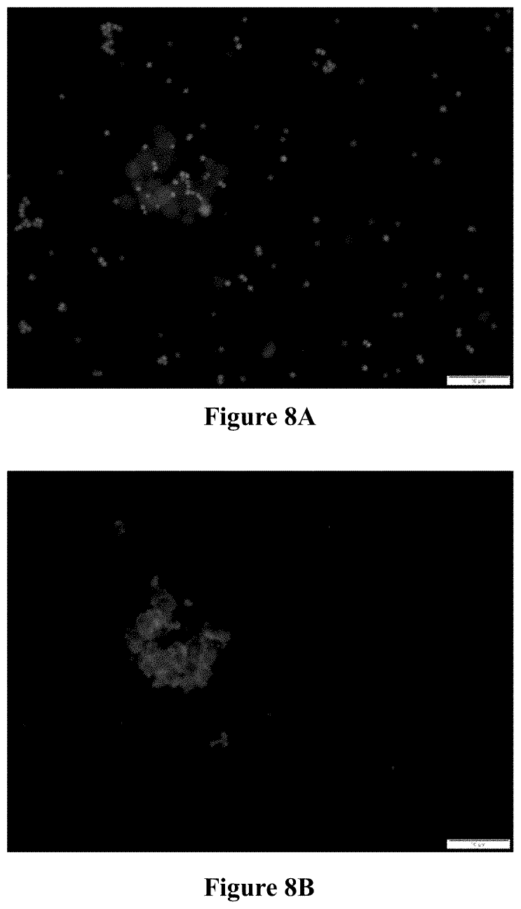

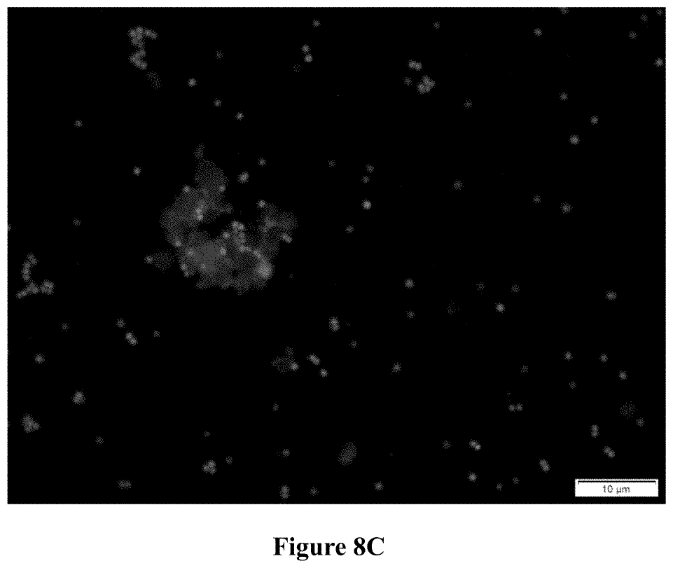

FIG. 8 shows that Dylight633-labeled thrombosomes in composition (3) co-agglutinated with BODIPY-labeled PANSORBIN® in the presence of purified 1 mg/mL human plasma fibrinogen and 1 mM Ca²⁺/Mg². FIG. 8A shows that PANSORBIN® cells appeared as punctate bodies in the GFP fluorescence channel. Faint thrombosomes autofluorescence in composition (3) was visible but can be discriminated from the PANSORBIN® signal by intensity. FIG. 8B shows that surface-stained thrombosomes in composition (3) aggregates were visible in the TexasRed fluorescence channel. Aggregates can be discriminated from non-aggregated thrombosomes in composition (3) by size. FIG. 8C shows that colocalization of PANSORBIN® and thrombosomes in composition (3) was apparent in the fluorescence overlay image. PANSORBIN® appeared bound to the surface of composition (3) in the larger aggregate. The physical interactions observed in this image were reproducible with (n=6) and without (n=2) supplemental fibrinogen and cations in the medium; without fibrinogen and cations only small aggregates are observed, whereas extremely large flakes form in the supplemented medium.

While the embodiments of the invention are amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

1. A method of treating an infection in a subject, the method comprising administering to the subject in need thereof an effective amount of a composition comprising platelets or platelet derivatives and an incubating agent comprising one or more salts, a buffer, optionally a cryoprotectant, and optionally an organic solvent.

2. The method of claim 27, wherein the bacterial infection is an antibiotic resistant bacterial infection.

3. (canceled)

4. The method of claim 27, wherein the viral infection is caused by a member of one or more of the following families: Adenoviridae, Herpesviridae, Papillomaviridae, Polyomaviridae, Poxviridae, Hepadnaviridae, Parvoviridae, Astroviridae, Caliciviridae, Picornaviridae, Coronaviridae, Flaviviridae, Togaviridae, Hepeviridae, Retroviridae, Orthomyxoviridae, Arenaviridae, Bunyaviridae, Filoviridae, Paramyxoviridae, Rhabdoviridae, or Reoviridae.

5. The method of claim 27, wherein the viral infection is caused by a human immunodeficiency virus (HIV).

6. The method of claim 27, wherein the viral infection is caused by a member of the Coronaviridae family.

7. (canceled)

8. The method of claim 6, wherein the the member of the Coronaviridae family is selected from the group consisting of SARS-CoV, MERS-CoV, SARS-CoV-2, or a combination thereof.

9. The method of claim 27, wherein the viral infection is caused by is Ebola virus, Marburg Virus, Lassa virus, or Dengue virus.

10. A method of binding a foreign body in a subject, the method comprising administering to the subject in need thereof an effective amount of a composition comprising platelets or platelet derivatives and an incubating agent comprising one or more salts, a buffer, optionally a cryoprotectant, and optionally an organic solvent, wherein the foreign body binds to the platelets or platelet derivatives.

11. (canceled)

12. The method of claim 10, wherein the foreign body is a bacterium, a fungus, a protozoa, or a virus.

13. (canceled)

14. (canceled)

15. (canceled)

16. The method of any one of claims 10, wherein the foreign body is in the bloodstream of the subject.

17. The method of any one of claims 10, wherein the foreign body is on the skin of the subject.

18. The method of any one of claims 1, wherein the platelets or platelet derivatives have been freeze-dried.

19. The method of any one of claims 1, wherein the incubating agent comprises one or more salts selected from phosphate salts, sodium salts, potassium salts, calcium salts, magnesium salts, and a combination of two or more thereof.

20. The method of any one of claims 1, wherein the buffer comprises HEPES, sodium bicarbonate (NaHCO₃), or a combination thereof.

21. The method of any one of claims 1, wherein the composition comprises one or more saccharides.

22. The method of claim 21, wherein the one or more saccharides comprise trehalose.

23. The method of claim 21, wherein the one or more saccharides comprise polysucrose.

24. The method of any one of claims 1, wherein administering comprises administering topically.

25. The method of any one of claims 1, wherein administering comprises administering intravenously.

26. The method of any one of claims 1, wherein administering comprises administering to the pulmonary system.

27. The method of claim 1, wherein the infection is a bacterial infection or a viral infection.
